(19) United States
(12) Patent Application Publication
(10) Pub. No.: US 2024/0175879 A1
(43) Pub. Date: May 30, 2024

(54) METHOD FOR ASSESSING EFFICACY OF TREATMENT PROTOCOLS FOR NEURODEGENERATIVE DISEASES

(71) Applicant: EM SCIENTIFIC LIMITED, London (GB)

(72) Inventors: Ernestas SIRKA, London (GB); Adam CRYAR, London (GB); Henri HUTTUNEN, London (GB)

(21) Appl. No.: 18/549,236

(22) PCT Filed: Mar. 8, 2022
(86) PCT No.: PCT/EP2022/055919

§ 371 Date: Sep. 6, 2023

(30) Foreign Application Priority Data
Mar. 8, 2021 (GB) 2103219.8

(51) Int. Cl. G01N 33/68

(52) U.S. Cl. CPC G01N 33/6896; G01N 33/6848; G01N 2800/2835; G01N 2800/52

(57) ABSTRACT

The present invention relates to a method for assessing the efficacy a treatment for a neurodegenerative disease in a subject wherein the presence of said proteolytic peptide is assayed for using mass spectrometry with reference to a corresponding labelled and/or unlabelled reference proteolytic peptide. The invention also relates to a method for stratifying a subject having a neurodegenerative disease, a method for the diagnosis of a neurodegenerative disease, methods for the treatment of a subject having a neurodegenerative disease and a kit for assessing the efficacy of a first-line treatment for a neurodegenerative disease in a subject.

The present invention relates to a method for assessing the efficacy of protocols for the treatment of neurodegenerative diseases using a multiplexed targeted proteomics-based assay.

Neurodegenerative diseases are a heterogeneous group of conditions that are characterised by the progressive degeneration of the function and structure of the peripheral nervous system or the central nervous system (CNS). The effective treatment of such diseases is increasingly important since the prevalence of the conditions is increasing in many countries. One route to study the course of treatment for such diseases is to look at biomarkers that can be obtained readily in biological samples from patients (Heywood et al., Molecular Neurodegeneration, 10, 64 (2015)).

Currently ELISA, SIMOA and other immunoassays are often used to measure a limited number of protein biomarkers in neurodegenerative diseases. Discovery proteomics may be used to measure multiple proteins at the same time although all of these technology platforms have significant limitations.

There are two main technical problems to be overcome. Firstly, the lack of comprehensive biomarker panels which would measure multiple neurodegenerative disease-relevant biomarkers. Secondly, the creation of a technology platform that would measure these multiple biomarkers in an accurate and reproducible way in a single test. Existing analytical technology platforms lack accuracy, reproducibility and/or multiplexing capacity.

Speed of deployment is another problem. It could take 6 to 9 months to raise new antibodies in addition to the time required to build an immunoassay once the antibody is available. Targeted proteomics assays can be built in 3-4 months which is an acceptable lead time for most clinical trials.

The previous work by Heywood et al. (2015) showed the utility of targeted proteomics in Parkinson's disease in the identification of biomarkers for the disease. However, the paper does not describe the concept of identifying biomarkers for assessing the efficacy of any given treatment protocol for Parkinson's disease.

The present invention provides a method for assessing the efficacy of a treatment protocol for a neurodegenerative disease using a multiplexed targeted proteomics assay. Suitably, the biomarkers are obtained from cerebrospinal fluid (CSF). The test provided by the invention can therefore monitor the treatment efficacy of new therapies currently in development for neurodegenerative diseases, such as Parkinson's disease.

According to a first aspect of the invention there is provided a method for assessing the efficacy of a treatment for a neurodegenerative disease in a subject, the method comprising:
(i) preparing a biological sample from the subject for assay by incubating the sample with a protease to form a proteolytic digest of proteins in the sample; and
(ii) assaying the proteolytic digest of proteins of step (i) for the presence of a proteolytic peptide of at least one protein selected from the group of proteins as shown in Table 1, said group consisting of Matrix metalloproteinase-9, Chitinase-3-like protein 1, Protein S100-A8, Protein S100-A9, Neutrophil collagenase (MMP8). Complement C3, Galectin-3-binding protein, Catalase, Extracellular superoxide dismutase, Prosaposin, Beta-hexosaminidase subunit beta, Cathepsin D, Beclin-1, Lysosome-associated membrane glycoprotein 1, Autophagy protein 13, E3 ubiquitin-protein ligase RNF26, E3 ubiquitin-protein ligase TRIM33, FAST kinase domain-containing protein 5, mitochondrial, Apoptosis-inducing factor 1 mitochondrial, Transmembrane protein 126A, Neurofilament light polypeptide, Chromogranin-A, Contactin-1, Neurexin-1-beta, Protein MTSS 1 (Metastasis suppressor YGL-1), Synaptotagmin-3, Apolipoprotein E, Apolipoprotein D, Clusterin, Flotillin-2, Cadherin-2, Neural cell adhesion molecule 2, Amyloid-beta precursor protein, Amyloid-like protein 1, Peptidyl-prolyl cis-trans isomerase FKBP3, Peptidyl-prolyl cis-trans isomerase FKBP4, Kallikrein-6, Neutrophil gelatinase-associated lipocalin, Protein disulfide-isomerase A1 (PDIA1), Limbic system-associated membrane protein (Fragment), Ceruloplasmin, Serotransferrin, Vacuolar protein sorting-associated protein 16 homolog (hVPS16), Peptidyl-prolyl cis-trans isomerase (PPlase; HEL-S-39), Lymphocyte cytosolic protein 2, Lactadherin, 72 kDa type IV collagenase, Transforming growth factor-beta-induced protein ig-h3, Neural proliferation differentiation and control protein 1, Ganglioside GM2 activator,
wherein the presence of said proteolytic peptide is assayed for using mass spectrometry with reference to a corresponding labelled and/or unlabelled reference proteolytic peptide.

The treatment for the neurodegenerative disease may comprise the administration of a pharmaceutical composition comprising a pharmaceutically active substance. The administration of the pharmaceutical composition may be part of a prescribed course of treatment or a clinical trial. The pharmaceutical composition may be administered once a day, twice a day, once a week, or at other frequencies according to the course of treatment prescribed or the clinical trial protocol. The clinical trial may be for one month, two months, 6-months or 12 months.

The efficacy of a treatment may be a measure of the effectiveness of any generally suitable method treatment for the neurodegenerative disease. The efficacy may be expressed in terms of the presence or absence of one or more clinical symptoms or one or more side-effects of the pharmaceutical composition used to treat the subject. The efficacy of a treatment may also therefore be correlated with a change in the concentration of a protein biomarker in a sample comprising a protein as shown in Table 1. The change in the concentration of a relevant biomarker as defined herein may be an increase in concentration of a biomarker or a decrease in concentration of a biomarker compared to a baseline or control value for such a biomarker.

Absolute peptide concentration may be compared across a set of proteins in a sample. The samples may be collected at different time intervals from the start of treatment regime (i.e. time at Week 0 (“Week zero”). Subsequent samples may be collected from patients at approximately Week 20 and/or Week 45. The results for the samples obtained at different time points can then be normalised to a baseline and the results can be expressed as relative change from the baseline. For example, a 1.5-fold peptide concentration increase or 0.67-fold concentration decrease from baseline in drug treated groups whilst having no or lesser corresponding peptide alteration in the placebo group may be considered to indicate drug treatment efficacy for a pharmaceutical composition comprising a drug under assessment according to a method of the invention.

Subsequent samples may be collected from patients at approximately Week 2, 4, 6, 8, 10, 12, 14, 16, 18, 20, 22, 24, 26, 28 or 30 and/or Week 35, 40, 45, 50, 55 or 60.

Subsequent samples may be collected from patients at approximately month 1, 2, 3, 4, 5 or 6 and/or month 7, 8, 9, 10, 11 or 12. Subsequent samples may be collected from patients at approximately six-month intervals. Subsequent samples may be collected from patients at approximately month 6 and/or month 12.

An at least 1.5-fold peptide concentration increase and/or an at least 0.67-fold concentration decrease from baseline in drug treated groups whilst having no or lesser corresponding peptide alteration in the placebo group may be considered to indicate drug treatment efficacy for a pharmaceutical composition comprising a drug under assessment according to a method of the invention.

An at least 1.1, at least 1.2, at least 1.3, at least 1.4, at least 1.5, at least 1.6, at least 1.7, at least 1.8, at least 1.9 or at least 2.0-fold peptide concentration increase and/or an at least 0.9, at least 0.8, at least 0.7, at least 0.6, at least 0.67, at least 0.5, at least 0.4, at least 0.3, at least 0.2 or at least 0.1-fold concentration decrease from baseline in drug treated groups whilst having no or lesser corresponding peptide alteration in the placebo group may be considered to indicate drug treatment efficacy for a pharmaceutical composition comprising a drug under assessment according to a method of the invention.

The neurodegenerative disease may be selected from the group consisting of Parkinson's Disease, Alzheimer's disease, Huntington's disease, Multiple Sclerosis (MS), Amyotrophic lateral sclerosis (ALS), Batten disease or a transmissible spongiform encephalopathy (e.g. scrapie, bovine spongiform encephalopathy (BSE) or Creutzfeldt-Jakob disease (CJD), (including iatrogenic, variant, familial or sporadic forms of CJD), or a lysosomal neurodegenerative disorder (e.g. Gaucher's disease). The neurodegenerative disease may be Parkinson's Disease.

As described herein, the methods of the present invention may be applicable to a broad range of other neurodegenerative conditions, such as Alzheimer's disease, and other disease that have a neurodegenerative component, for instance rare diseases/inborn errors of metabolism/lysosomal storage disorders etc. The test according to a method of the invention may also applicable to inflammatory conditions, for instance Cystatin C in kidney diseases.

The sample may be cerebrospinal fluid (CSF), blood (serum, whole blood (venous blood or peripheral blood), plasma, urine, tear, saliva, interstitial fluid, lymph fluid or tissue samples. The sample may be cerebrospinal fluid (CSF). The sample may be collected by any suitable means including collection of CSF by lumbar puncture, venous blood collection by direct removal from a vein or potentially via a finger prick blood collection device or a bloodspot card or plasma spot card.

The same proteins may also be measured using a different technology platform. ELISA, SIMOA, Olink, Western Blot, or other immunoassay platforms may be used to the measure the same protein set. Aptamers (oligonucleotide or peptide molecules that bind to a specific target molecule) can be used instead of antibodies in similar assays.

Different sample types may be used for this test. Currently, the test is validated on neat CSF samples but different biofluid samples, such as serum, plasma with its different sample additives (anticoagulants) may be suitable. This includes K2 and K3 EDTA plasma tubes, heparin, potassium oxalate/sodium fluoride treated plasma tubes and others.

The protease may be a serine protease (e.g. trypsin, chymotrypsin, thrombin, elastase, or subtilisin), a cysteine protease (e.g. papain, caspase-1, adenain, pyroglutamly-peptidase I, sortase A, hepatitis C virus peptidase 2, sindbis virus-type nsP2 peptidase, dipeptidyl-peptidase VI, DeSI-1 peptidase, TEV protease, amidophosphoribozyltransferase precursor, gamma-glutamyl hydrolase, hedgehog protein, dmpA aminopeptidease), a threonine protease (e.g. ornithine acetyltransferase), an aspartic protease (e.g. pepsin, cathepsin D, cathepsin E, napsin-A, nepenthesin, presenilin, renin (chymosin)), a glutamic protease (e.g. scytalidoglutamic peptidase (eqolisin), aspergilloglutamic peptidase), a metalloprotease (e.g. an ADAM or a matrix metalloproteinase), or an asparagine peptide lyase. The protease may be trypsin.

A proteolytic peptide is therefore a peptide sequence from a protein which has been produced by the action of a protease cleaving a peptide bond between amino acids in the protein sequence. Such proteolytic peptides are therefore oligopeptides being formed of a number of amino acids. The proteolytic peptide may be from 5 to 30 amino acid residues in length, suitably 6 to 20 amino acids in length, 7 to 25 amino acids in length, or any of 5, 6, 7, 8, 9, 10, 11, 12, 13, 14, 15, 16, 17, 18, 19, 20, 21, 22, 23, 24, 25, 26, 27, 28, 29 or 30 amino acids in length.

A proteolytic peptide prepared by the action of the protease trypsin on a protein may be referred to as a tryptic peptide and so on.

The proteolytic peptide may suitably have a sequence as set out in Table 1.

The method of the invention may comprise assaying for the presence of up to all 50 proteins in Table 1 with respect to a proteolytic peptide thereof. In some embodiments, the method of the invention may comprise assaying for the presence of a proteolytic peptide of at least 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 proteins as shown in Table 1.

The methods of the invention therefore comprise the assaying for up to 118 proteolytic peptides of the 50 proteins shown in Table 1. In some embodiments, the method of the invention may comprise assaying for at least 5 to 10, 5 to 15, 5 to 20, 5 to 25, 5 to 30, 5 to 35, 5 to 40, 5 to 45 or 5 to 50 proteolytic peptides of Table 1. Preferably the number of proteolytic peptides assayed for is at least 5, at least 15, at least 20 or at least 25 proteolytic peptides as shown in Table 1. The number of proteolytic peptides assayed for may be 48 proteolytic peptides as shown in Table 1. When 48 proteolytic peptides shown in Table 1 are assayed for, the proteolytic peptides may be one proteolytic peptide of each protein of Table 1 except for Protein MTSS 1 (Metastasis suppressor YGL-1) and Lymphocyte cytosolic protein 2.

When more than one protein and/or proteolytic peptide is assayed for, the method can be said to comprise assaying the proteolytic digest of proteins of step (i) for the presence of a panel of proteolytic peptides of at least two proteins selected from the group of proteins as shown in Table 1. Preferably, the method may comprise assaying the proteolytic digest of proteins of step (i) for the presence of a panel of proteolytic peptides of at least 48 proteins, wherein the 48 proteins are:
Matrix metalloproteinase-9
Chitinase-3-like protein 1
Protein S100-A8
Protein S100-A9
Neutrophil collagenase (MMP8)
Complement C3
Galectin-3-binding protein
Catalase
Extracellular superoxide dismutase
Prosaposin
Beta-hexosaminidase subunit beta
Cathepsin D
Beclin-1
Lysosome-associated membrane glycoprotein 1
Autophagy protein 13
E3 ubiquitin-protein ligase RNF26
E3 ubiquitin-protein ligase TRIM33
FAST kinase domain-containing protein 5, mitochondrial
Apoptosis-inducing factor 1, mitochondrial
Transmembrane protein 126A
Neurofilament light polypeptide
Chromogranin-A
Contactin-1
Neurexin-1-beta
Synaptotagmin-3
Apolipoprotein E
Apolipoprotein D
Clusterin
Flotillin-2
Cadherin-2
Neural cell adhesion molecule 2
Amyloid-beta precursor protein
Amyloid-like protein 1
Peptidyl-prolyl cis-trans isomerase FKBP3
Peptidyl-prolyl cis-trans isomerase FKBP4
Kallikrein-6
Neutrophil gelatinase-associated lipocalin
Protein disulfide-isomerase A1 (PDIA1)
Limbic system-associated membrane protein (Fragment)
Ceruloplasmin
Serotransferrin
Vacuolar protein sorting-associated protein 16 homolog (hVPS16)
Peptidyl-prolyl cis-trans isomerase (PPlase; HEL-S-39)
Lactadherin
72 kDa type IV collagenase
Transforming growth factor-beta-induced protein ig-h3
Neural proliferation differentiation and control protein 1 and
Ganglioside GM2 activator.

Table 1 shows 50 proteins differentially expressed in Parkinson's Disease patients following drug treatment and corresponding 118 proteolytic peptide sequences with corresponding heavy isotope-labelled peptide internal standard sequences. The methods of the invention may comprise Table 1 also shows haemoglobin subunit beta as an optional suitable analytical control protein with corresponding proteolytic peptide sequence and corresponding heavy isotope-labelled peptide internal standard sequence.

An internal standard sequence may not be required in order to analyse the expression of any given protein according to a method of the invention. A single protein internal standard can be used for the entire biomarker test, e.g. yeast enolase without any heavy isotope-labelled internal standards. Other heavy isotope-labelled internal standards having a similar retention time can be used for quantification of peptides which do not have their own internal standards. There are examples of targeted proteomics methods which use no internal standards at all, for example the University College Dublin test for prostate cancer.

In one embodiment, the method of the invention may suitably comprise assaying for the six proteolytic peptides in the following group of proteins and corresponding proteolytic peptides:

| Protein | Proteolytic peptide |
|---|---|
| Chromogranin-A | SGELEQEEER (SEQ ID |
| | No. 52) |
| Amyloid-beta Precursor | QQLVETHMAR (SEQ ID |
| Protein | No. 78) |
| Amyloid-like Protein 1 | QMYPELQIAR (SEQ |
| | ID No. 85) |
| Transforming Growth Factor- | GDELADSALEIFK (SEQ |
| Beta-Induced Protein IG-H3 | ID No. 114) |
| Prosaposin | QEILAALEK (SEQ ID |
| | No. 23) |
| Apolipoprotein E | AATVGSLAGQPLQER |
| | SEQ ID No. 64) |

The at least one protein may be selected from the group consisting of Chromogranin-A, Amyloid-beta Precursor Protein, Amyloid-like Protein 1, Transforming Growth Factor-Beta-Induced Protein IG-H3, Prosaposin and Apolipoprotein E. The at least one protein may be selected from the group consisting of Chromogranin-A, Amyloid-beta Precursor Protein, Amyloid-like Protein 1, Transforming Growth Factor-Beta-Induced Protein IG-H3 and Prosaposin.

The proteolytic peptide of at least one protein may be selected from the group consisting of:

(SEQ ID No. 52)
SGELEQEEER
(SEQ ID No. 78)
QQLVETHMAR
(SEQ ID No. 85)
QMYPELQIAR
(SEQ ID NO. 114)
GDELADSALEIFK
(SEQ ID No. 23)
QEILAALEK
and
(SEQ ID No. 64)
AATVGSLAGQPLQER.

The proteolytic peptide of at least one protein may be selected from the group consisting of:

(SEQ ID No. 52)
SGELEQEEER
(SEQ ID No. 78)
QQLVETHMAR
(SEQ ID No. 85)
QMYPELQIAR
(SEQ ID No. 114)
GDELADSALEIFK
and
(SEQ ID NO. 23)
QEILAALEK.

In one embodiment, the at least one protein is Chromogranin-A and the proteolytic peptide of the at least one protein is SGELEQEEER (SEQ ID No. 52).

In one embodiment, the at least one protein is Amyloid-beta Precursor Protein and the proteolytic peptide of the at least one protein is QQLVETHMAR (SEQ ID No. 78).

In one embodiment, the at least one protein is Amyloid-like Protein 1 and the proteolytic peptide of the at least one protein is QMYPELQIAR (SEQ ID No. 85). The internal standard may be QMYPELQIAR(U-13C6,15N4)VEQAT (SEQ ID No. 162).

In one embodiment, the at least one protein is Transforming Growth Factor-Beta-Induced Protein IG-H3 and the proteolytic peptide of the at least one protein is GDELADSALEIFK (SEQ ID No. 114).

In one embodiment, the at least one protein is Prosaposin and the proteolytic peptide of the at least one protein is QEILAALEK (SEQ ID No. 23). The internal standard may be QEILAALEK(U-13C6,15N2)GCSFL (SEQ ID No. 132).

In one embodiment, the at least one protein is Apolipoprotein E and the proteolytic peptide of the at least one protein is AATVGSLAGQPLQER (SEQ ID No. 64). The internal standard may be AATVGSAGQPLQER(U-13C6,15N4)AQAWG (SEQ ID No. 151).

The methods of the present invention use mass spectrometry to identify the concentrations of the peptides, for example proteolytic peptides having the sequences as set out in Table 1. In a mass spectrometry (MS) system for the analysis of proteolytic peptides in a sample, the proteolytic peptides are injected into the MS system where the proteolytic peptides are first detected as intact peptides and then subsequently fragmented into smaller pieces which may be termed peptide fragments. The methods of the present invention provide for the detection of up to 118 proteolytic peptides of the up to 50 proteins as shown in Table 1. The concentrations of the peptides may be used to assess the efficacy of a treatment for a neurodegenerative disease and/or to stratify a subject having a neurodegenerative disease, with reference to a calibration curve of the concentrations for known reference proteolytic peptides.

The concentrations of the proteolytic peptides assayed for in the samples are therefore linked with assessing the efficacy of a treatment for a neurodegenerative disease and/or stratifying a subject having a neurodegenerative disease. Generally, a difference in proteolytic peptide concentration from a control (either increased or decreased), indicates efficacy of a treatment for a neurodegenerative disease and/or stratifying a subject having a neurodegenerative disease.

The control may be a baseline control. The control may be a placebo group. The concentrations of the proteolytic peptides may be normalised to the control. Concentrations may be expressed as fold changes relative to the control, such as fold changes from baseline.

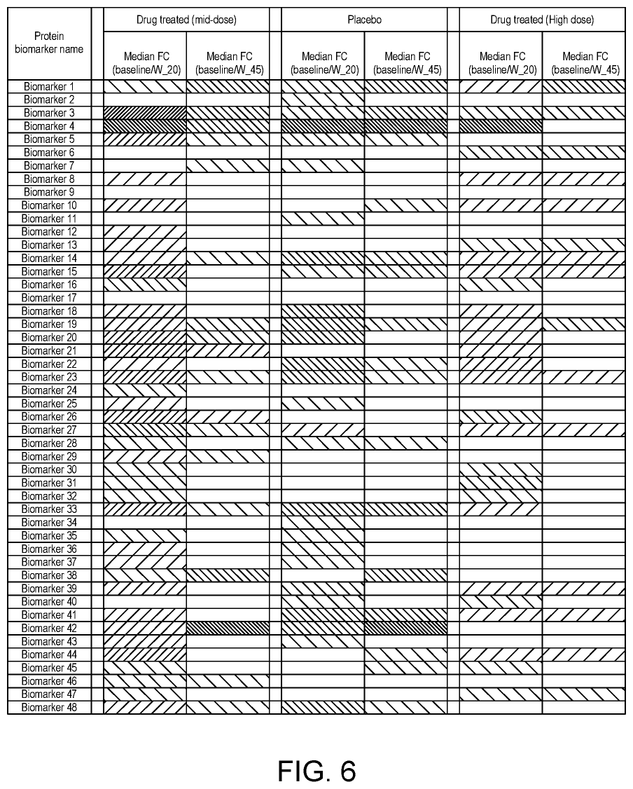

The methods of the invention may further account for whether the proteolytic peptides are shown herein to be up-regulated or downregulated. Biomarker levels across different patients groups, for instance in a clinical trial, may be different; i.e. increased or decreased relative to another group (such as a control or a placebo). A combination of biomarkers may be used to separate patient groups based on biomarker concentration and using a principal component analysis (PCA) or fold change differences. Fold change differences may be used to separate patient groups using a threshold or cut-off point, for example as illustrated FIGS. 6 & 7.

Reference peptides may be used to pre-configure the mass spectrometer prior to use in a method of the invention to detect and quantitate the concentration of peptides of Table 1 in a sample. The reference peptides also allow for the construction of calibration lines with each batch of samples tested in order to ensure robust results. Heavy isotope-labelled peptides may be used as internal standards to control analytical variability in each sample and also provide for calibration lines.

According to the present invention a mass spectrometry platform is used to measure the concentration of the analytes. Whilst triple quadrupole mass spectrometry platforms, operated in MRM mode, are preferred for this test, there could be other mass spectrometry platforms or other data acquisition modes used to design around this test. One example is high resolution mass spectrometry platforms (e.g. Sciex 6600, Thermo Orbitrap or Waters QTOF-type instruments) where “pseudo” MRM or PRM modes could be used for measurements. Triple quadrupole instruments are more robust and provide more reproducible data and thus other platforms may not reach the same performance in all aspects of the test.

There is also a variety of chromatography systems which can be coupled to a mass spectrometry platform. The test is designed to use “standard flow” (ml/min) liquid chromatography systems but lower flow systems may be used—in a range of μl/min or nl/min.

Peptide ionisation interface/method prior to mass spectrometry analysis could also be different. The test is designed to use electrospray ionisation (ESI), however the peptides can be ionised using matrix-assisted laser desorption/ionization (MALDI) or desorption electrospray ionization (DESI) or atmospheric-pressure chemical ionization (APCI) or other ionisation methods.

Different internal standards on the same targeted proteomics platform can be used. The test is designed to use heavy isotope-labelled internal standards for accurate measurements, however differently labelled or unlabelled peptide or protein internal standards could be used which may or may not meet the performance of heavy isotope-labelled standards.

The method may comprise determining the relative concentration of the proteolytic peptide measured in the sample. The relative concentration of a peptide measured in the sample may refer to the concentration of the proteolytic peptide relative to the corresponding labelled and/or unlabelled reference proteolytic peptide.

Any of the tests described herein may be multiplexed with another test measuring proteins in a biological sample, e.g. CSF proteins, with the same technology platform. Thus, the test of the present invention may be a part of a larger test. If other treatment efficacy tests emerge, the test of the present invention could be used to augment such other tests to enhance overall performance.

The proteins from which the peptide sequences are derived are set out in Table 1 described herein. The method of the present invention may be a multiplexed assay.

As set out herein, a different peptide set to that shown in Table 1 could also be measured from the same proteins as shown in Table 1 using the same targeted proteomics platform but using a different protease. The examples of the present invention described herein show one embodiment of a method of the invention, however different peptides from the same set of 50 proteins may be used. This includes peptides generated using the same protease as in this test (trypsin) or different proteases (LysC, GluC etc).

Typically, the mass spectrometry analysis of peptides according to a method of the present invention, is liquid chromatography-targeted mass spectrometry (LC-MS) using triple quadrupole instruments, operated in timed multiple reaction monitoring (MRM) mode.

The methods of the present invention therefore comprise the use of targeted proteomics. The technique comprises the quantification of specific, pre-selected proteins or proteolytic peptides from a given sample and requires a pre-existing understanding of disease biology to guide protein selection. The technique is therefore distinct from discovery proteomics which seeks to gather information about all proteins and proteolytic peptides in a sample without pre-existing knowledge/hypotheses around disease biology. Internal standards or calibration lines for every protein or peptide of interest are not and cannot be used in discovery proteomics. Discovery proteomics is also conducted using different instrument operation modes, e.g. SWATH, HDMSE etc comparted to MRM or PRM in targeted proteomics. Data processing also uses different approach to signal normalisation and quantification where absolute concentration cannot be provided. Discovery proteomics platforms lack robustness and reproducibility of targeted proteomics platforms. Targeted and discovery proteomics are distinct to the extent that a team of scientists utilising discovery proteomics platforms are generally not able to develop a targeted proteomics biomarker test without specific knowledge and experience in targeted proteomics. This is due to the above mentioned and other differences at every stage of the process, from initial concepts, sample preparation, data acquisition and processing to final test implementation in a clinical setting.

The methods of the invention are performed using a proteolytic reference peptide and may be configured to use any suitable internal standard peptide on the same targeted proteomics platform. The methods of the invention may suitably be used with heavy isotope-labelled internal standards for accurate measurements. Examples of internal standards as heavy-isotope labelled proteolytic peptides are shown in Table 1 with respect to the proteolytic peptides described therein. The internal standard may be added to the sample before a proteolytic digestion of the proteins in the sample has occurred in step (i) of the methods of the invention. Alternatively, internal standard may be added to the sample after a proteolytic digestion of the proteins in the sample has occurred in step (i) of the methods of the invention. The mass spectrometer may be pre-configured using said internal standard. Suitable, heavy-isotope labels are ¹³C, ¹⁵N; and/or ²H.

In one embodiment the method of the invention may be used to assess the efficacy of a treatment regime for Parkinson's Disease. The test measures the concentration of 118 peptides, arising from 50 CSF proteins as detailed in Table 1. Several proteins described herein were not previously associated with Parkinson's disease and were not reported as biomarkers of drug treatment efficacy in Parkinson's disease (PD). This protein set covers all biochemical aspects of Parkinson's disease pathology in a single assay which include: Inflammation; Oxidative stress; Autophagy/Lysosomal; Ubiquitin Proteasome System (UPS); Mitochondrial; Axonal/neuronal degeneration; Synaptic degeneration; Lipoprotein metabolism; Enhanced release of exosomes; Endothelial dysfunction; Amyloid Processing and other proteins relevant to PD. The test also includes haemoglobin as a control to monitor for CSF contamination with blood.

The methods of the present invention may comprise steps performed by a computer and involve equipment controlled by the computer. The step of assaying the proteolytic digest of proteins of step (i) for the presence of a proteolytic peptide may be performed by equipment controlled by the computer.

The invention also provides a computer-implemented method for assessing the efficacy of a treatment for a neurodegenerative disease in a subject, which comprises receiving in a computer sample data representing the level of at least one proteolytic peptide in sample obtained from a subject and executing software on the computer to compare the level of the at least at least one proteolytic peptide in the sample to a baseline control, wherein the difference between the level of the at least one proteolytic peptide and the baseline control is indicative of the efficacy of a treatment for a neurodegenerative disease, and to output efficacy data representing the efficacy of a treatment for a neurodegenerative disease on the basis of the comparison.

The invention also provides a computer program comprising instructions which, when executed by a computer, cause the computer to carry out a computer implemented method of the invention.

It will be appreciated that the step of comparing the level of the at least at least one proteolytic peptide in the sample with a baseline control may be carried out on a different computer from a computer that initially receives data representing the at least at least one proteolytic peptide in the sample.

The invention also provides a computer apparatus for assessing the efficacy of a treatment for a neurodegenerative disease, which comprises a first device incorporating a computer, a second computer and a communication channel between the first device and second computer for the transmission of data therebetween; wherein the first device is arranged to receive sample data representing level of the at least one proteolytic peptide in a sample obtained from the subject and to transmit the sample data to the second computer via the communication channel, and the second computer is arranged to execute software to compare levels of the at least at least one proteolytic peptide in the sample to a baseline control to determine the efficacy of a treatment for a neurodegenerative disease, wherein the difference between the level of the at least one proteolytic peptide and the baseline control is indicative of the efficacy of a treatment for a neurodegenerative disease, and to output efficacy data representing the efficacy of a treatment for a neurodegenerative disease on the basis of the comparison.

The second computer may be arranged to transmit the efficacy data to the first device via the communication channel, or to a third computer.

In some embodiments, the first device may incorporate mass spectrometry equipment or devices for measuring the level of at least one proteolytic peptide in a sample.

The test is built on the only type of mass spectrometry platform which can be accredited to existing regulatory standards and is suitable for ongoing clinical testing. Uniquely, the test measures 118 peptides at the same time in the most robust way possible as every peptide measured has a dedicated calibration line and an internal standard to provide absolute concentration and to correct for analytical signal variability. CSF sample preparation protocol has also been experimentally optimised to detect this specific set of proteins.

The test allows rapid iteration of the protein set where newly discovered molecules can be included. This also allows for this assay to be tailored to a specific therapeutic, patient population or a neurodegenerative disease (sub)type.

The present invention provides the first comprehensive targeted proteomics biomarker test to assess treatment efficacy of new therapies in development for neurodegenerative diseases, such as Parkinson's disease, globally.

This test system provided by the present invention can be commercialised in a standardised “assay kit” format which will provide end-to-end solution for customers to set up in their own laboratories in addition to sending samples to a centralised testing facility.

The methods of the present invention may be used as a biofluid test to assess treatment efficacy of novel therapies in development or existing/repurposed treatments for the treatment of a neurodegenerative disease, for example Parkinson's Disease. Such uses include therapies in clinical, pre-clinical development and pharmacovigilance.

FIG. 6 shows concentration changes of 48 biomarkers. It indicates that the biomarker signature across 3 patient groups (placebo, drug treated mid-dose and drug-treated high dose) is different: in placebo group most biomarkers decrease over time, whereas in both drug treated groups the biomarkers mostly increase over time. Thus, using any number of biomarkers from this panel of 48 biomarkers it is possible to detect molecular level changes after drug treatment in a placebo controlled randomised clinical trial, indicating the application of the invention as a tool to assess drug treatment effects.

The methods of the present invention may be used as a biofluid test to stratify subjects having a neurodegenerative disease, e.g. Parkinson's disease patient populations, according to response to therapy. Such methods would be a companion diagnostics or complementary diagnostics test or a tool for precision medicine used on clinical trials, pre-clinical studies or in healthcare settings after therapy approval.

According to second aspect of the invention, there is provided a method for stratifying a subject having a neurodegenerative disease, the method comprising:
(i) preparing a biological sample from the subject for assay by incubating the sample with a protease to form a proteolytic digest of proteins in the sample; and
(ii) assaying the proteolytic digest of proteins of step (i) for the presence of a proteolytic peptide of at least one protein selected from the group of proteins as shown in Table 1, said group consisting of Matrix metalloproteinase-9, Chitinase-3-like protein 1, Protein S100-A8, Protein S100-A9, Neutrophil collagenase (MMP8). Complement C3, Galectin-3-binding protein, Catalase, Extracellular superoxide dismutase, Prosaposin, Beta-hexosaminidase subunit beta, Cathepsin D, Beclin-1, Lysosome-associated membrane glycoprotein 1, Autophagy protein 13, E3 ubiquitin-protein ligase RNF26, E3 ubiquitin-protein ligase TRIM33, FAST kinase domain-containing protein 5, mitochondrial, Apoptosis-inducing factor 1 mitochondrial, Transmembrane protein 126A, Neurofilament light polypeptide, Chromogranin-A, Contactin-1, Neurexin-1-beta, Protein MTSS 1 (Metastasis suppressor YGL-1), Synaptotagmin-3, Apolipoprotein E, Apolipoprotein D, Clusterin, Flotillin-2, Cadherin-2, Neural cell adhesion molecule 2, Amyloid-beta precursor protein, Amyloid-like protein 1, Peptidyl-prolyl cis-trans isomerase FKBP3, Peptidyl-prolyl cis-trans isomerase FKBP4, Kallikrein-6, Neutrophil gelatinase-associated lipocalin, Protein disulfide-isomerase A1 (PDIA1), Limbic system-associated membrane protein (Fragment), Ceruloplasmin, Serotransferrin, Vacuolar protein sorting-associated protein 16 homolog (hVPS16), Peptidyl-prolyl cis-trans isomerase (PPlase; HEL-S-39), Lymphocyte cytosolic protein 2, Lactadherin, 72 kDa type IV collagenase, Transforming growth factor-beta-induced protein ig-h3, Neural proliferation differentiation and control protein 1, Ganglioside GM2 activator,
wherein the presence of said proteolytic peptide is assayed for using mass spectrometry with reference to a corresponding labelled and/or unlabelled reference proteolytic peptide; and
(b) stratifying the subject according to the relative concentration of a peptide measured in the sample.

To stratify patient groups after drug treatment, biomarker (i.e. peptide) trajectories within a drug treated patient group may be compared. All patients with biomarker signal increase (i.e. an increase in concentration of the biomarker protein being assayed for) may be stratified into a first subgroup whereas all patients with biomarker signal decrease or not significantly changing biomarker signal (i.e. a decrease in concentration of the biomarker protein being assayed for or no change in concentration) may be classified into a second subgroup. The change in concentration (either increase or decrease) may be with respect to a baseline control value or measured over a period of time (e.g. 20 weeks or 45 weeks).

The relative concentration of a peptide measured in the sample may refer to the concentration of the peptide relative to the corresponding labelled and/or unlabelled reference proteolytic peptide.

Stratifying the subject according to the relative concentration of a peptide measured in the sample may comprise separating the subjects into two or more groups according to the relative concentration of a peptide measured in the sample. The relative concentration used for stratifying and/or grouping the subjects may be at any suitable time point, such as baseline, week 20 and/or week 45 after drug treatment. Any suitable threshold may be used for stratifying and/or grouping the subjects. For example, the subjects may be stratified into two groups based on having a relative concentration of a peptide which is either above or below the average relative concentration of the peptide for all subjects being stratified. The average may be the mean, the median or the mode. Alternatively, the subjects may be stratified into two groups based on having a relative concentration of a peptide which is either above or below a threshold value. The threshold value may be predetermined. The threshold value may be any suitable value such as 0.02, 0.03, 0.04, 0.05, 0.06, 0.07, 0.08, 0.09, 0.10, 0.11, 0.12, 0.13, 0.14 or 0.15.

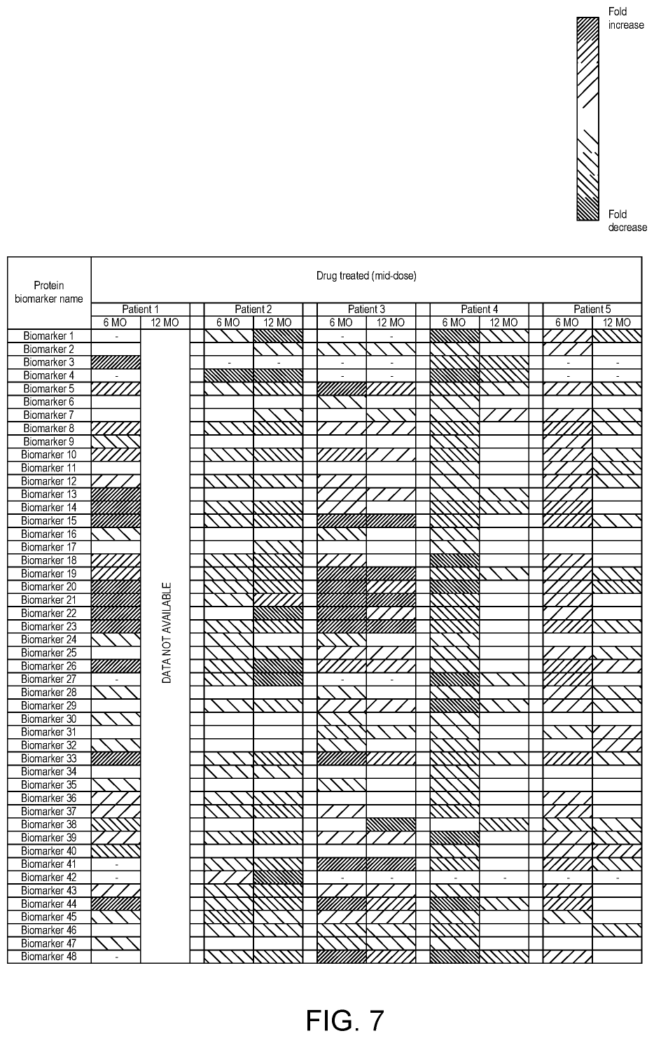

FIG. 7 shows concentration changes of 48 biomarkers over time in 5 patients who were diagnosed with Parkinson's Disease and were treated with a novel Parkinson's Disease drug in clinical development. The figure shows biomarker concentration fold change from baseline at 6 and 12 months which is different within this patient group, i.e. Patients 2 and 4 respond differently (most biomarkers decreased after treatment) to patients 3 and 5 (biomarkers increase after treatment). Current expert consensus in the Parkinson's Disease field is that Parkinson's Disease may be a heterogenous disease at both clinical and molecular level and hence patients may respond differently to drug treatment. Further, this specific drug is expected to elicit different responses in different Parkinson's Disease patients as a result of its multimodal mechanism of action. Thus, the biomarker results obtained and shown in this figure indicate the invention provides a practical tool (for instance a CSF biomarker test) to identify these different patient groups, and therefore finds application in patient stratification.

The method may comprise assaying the proteolytic digest of proteins of step (i) for the presence of a proteolytic peptide of at least one protein selected from the consisting of E3 Ubiquitin-Protein Ligase TRIM33, Fast Kinase Domain-Containing Protein 5, Mitochondrial and Contactin-1. The proteolytic peptide of E3 Ubiquitin-Protein Ligase TRIM33 may be LFCETCDR (SEQ ID No. 40). The proteolytic peptide of Fast Kinase Domain-Containing Protein 5, Mitochondrial may be LAVQFTNR (SEQ ID No. 41). The proteolytic peptide of Contactin-1 may be IVESYQIR (SEQ ID No. 54).

The method may comprise assaying the proteolytic digest of proteins of step (i) for the presence of a proteolytic peptide of at least one protein wherein the protein is E3 Ubiquitin-Protein Ligase TRIM33 and the proteolytic peptide is LFCETCDR (SEQ ID No. 40).

The method may comprise assaying the proteolytic digest of proteins of step (i) for the presence of a proteolytic peptide of at least one protein wherein the protein Fast Kinase Domain-Containing Protein 5, Mitochondrial and the proteolytic peptide is LAVQFTNR (SEQ ID No. 41).

The method may comprise assaying the proteolytic digest of proteins of step (i) for the presence of a proteolytic peptide of at least one protein wherein the protein is Contactin-1 and the proteolytic peptide is IVESYQIR (SEQ ID No. 54).

The methods of the present invention may be used as biofluid test to help assess safety of novel therapies in development or existing treatments for neurodegenerative diseases, for example Parkinson's Disease. This includes therapies in clinical or pre-clinical development.

The methods of the present invention may be used as biofluid test to compare severity and other aspects of neurodegenerative diseases, for example Parkinson's disease, as a result of different aetiology (different genetic mutations, sporadic, early onset, different toxins, as a result of other conditions e.g. Gaucher disease etc).

The methods of the present invention may be used as biofluid test for population studies to determine which sub-groups of the population develop milder or more severe Parkinson's disease. This includes age, different geographic locations, diet, comorbidities etc.

The methods of the present invention may be used as biofluid test to help elucidate the mechanism of action of new therapies in development. The methods of the present invention may be used as biofluid test as a reference method/benchmark for future tests for neurodegenerative diseases, for example Parkinson's Disease, which includes cross-validation at test development stages.

Parts of this test described herein may potentially be used in a future diagnostic test for a neurodegenerative disease, for example Parkinson's disease. Parts of this test described herein may potentially be used to stratify Parkinson's disease patient population according to disease stage/severity in clinical settings. Parts of this test described herein may potentially be used to as a prognostic/predictive tool to determine an individual's risk to develop a neurodegenerative disease, for example Parkinson' Disease, or a more serve form/stage of the disease

According to third aspect of the invention, there is provided a method for the diagnosis of a neurodegenerative disease, the method comprising:
(i) preparing a biological sample from the subject for assay by incubating the sample with a protease to form a proteolytic digest of proteins in the sample; and
(ii) assaying the proteolytic digest of proteins of step (i) for the presence of a proteolytic peptide of at least one protein selected from the group of proteins as shown in Table 1, said group consisting of Matrix metalloproteinase-9, Chitinase-3-like protein 1, Protein S100-A8, Protein S100-A9, Neutrophil collagenase (MMP8). Complement C3, Galectin-3-binding protein, Catalase, Extracellular superoxide dismutase, Prosaposin, Beta-hexosaminidase subunit beta, Cathepsin D, Beclin-1, Lysosome-associated membrane glycoprotein 1, Autophagy protein 13, E3 ubiquitin-protein ligase RNF26, E3 ubiquitin-protein ligase TRIM33, FAST kinase domain-containing protein 5, mitochondrial, Apoptosis-inducing factor 1 mitochondrial, Transmembrane protein 126A, Neurofilament light polypeptide, Chromogranin-A, Contactin-1, Neurexin-1-beta, Protein MTSS 1 (Metastasis suppressor YGL-1), Synaptotagmin-3, Apolipoprotein E, Apolipoprotein D, Clusterin, Flotillin-2, Cadherin-2, Neural cell adhesion molecule 2, Amyloid-beta precursor protein, Amyloid-like protein 1, Peptidyl-prolyl cis-trans isomerase FKBP3, Peptidyl-prolyl cis-trans isomerase FKBP4, Kallikrein-6, Neutrophil gelatinase-associated lipocalin, Protein disulfide-isomerase A1 (PDIA1), Limbic system-associated membrane protein (Fragment), Ceruloplasmin, Serotransferrin, Vacuolar protein sorting-associated protein 16 homolog (hVPS16), Peptidyl-prolyl cis-trans isomerase (PPlase; HEL-S-39), Lymphocyte cytosolic protein 2, Lactadherin, 72 kDa type IV collagenase, Transforming growth factor-beta-induced protein ig-h3, Neural proliferation differentiation and control protein 1, Ganglioside GM2 activator,
wherein the presence of said proteolytic peptide is assayed for using mass spectrometry with reference to a corresponding labelled and/or unlabelled reference proteolytic peptide; and
(b) diagnosing the neurodegenerative disease of the subject according to the relative concentration of a peptide measured in the sample.

According to fourth aspect of the invention, there is provided a method for the treatment of a subject having a neurodegenerative disease, the method comprising:
(a) (i) preparing a biological sample from the subject for assay by incubating the sample with a protease to form a proteolytic digest of proteins in the sample; and
(ii) assaying the proteolytic digest of proteins of step (i) for the presence of a proteolytic peptide of at least one protein selected from the group of proteins as shown in Table 1, said group consisting of Matrix metalloproteinase-9, Chitinase-3-like protein 1, Protein S100-A8, Protein S100-A9, Neutrophil collagenase (MMP8). Complement C3, Galectin-3-binding protein, Catalase, Extracellular superoxide dismutase, Prosaposin, Beta-hexosaminidase subunit beta, Cathepsin D, Beclin-1, Lysosome-associated membrane glycoprotein 1, Autophagy protein 13, E3 ubiquitin-protein ligase RNF26, E3 ubiquitin-protein ligase TRIM33, FAST kinase domain-containing protein 5, mitochondrial, Apoptosis-inducing factor 1 mitochondrial, Transmembrane protein 126A, Neurofilament light polypeptide, Chromogranin-A, Contactin-1, Neurexin-1-beta, Protein MTSS 1 (Metastasis suppressor YGL-1), Synaptotagmin-3, Apolipoprotein E, Apolipoprotein D, Clusterin, Flotillin-2, Cadherin-2, Neural cell adhesion molecule 2, Amyloid-beta precursor protein, Amyloid-like protein 1, Peptidyl-prolyl cis-trans isomerase FKBP3, Peptidyl-prolyl cis-trans isomerase FKBP4, Kallikrein-6, Neutrophil gelatinase-associated lipocalin, Protein disulfide-isomerase A1 (PDIA1), Limbic system-associated membrane protein (Fragment), Ceruloplasmin, Serotransferrin, Vacuolar protein sorting-associated protein 16 homolog (hVPS16), Peptidyl-prolyl cis-trans isomerase (PPlase; HEL-S-39), Lymphocyte cytosolic protein 2, Lactadherin, 72 kDa type IV collagenase, Transforming growth factor-beta-induced protein ig-h3, Neural proliferation differentiation and control protein 1, Ganglioside GM2 activator,
wherein the presence of said proteolytic peptide is assayed for using mass spectrometry with reference to a corresponding labelled and/or unlabelled reference proteolytic peptide;
(b) diagnosing the neurodegenerative disease of the subject according to the relative concentration of a peptide measured in the sample; and
(c) administering a pharmaceutical composition to the subject for the treatment of the neurodegenerative disease.

Suitable pharmaceutical compositions for the treatment of Parkinson's Disease included, but are not limited to: bromocriptine, cabergoline, lazabemide, levodopa, pergolide, pramipexole, rasagiline, ropinirole, rotigotine and selegiline.

Suitable pharmaceutical compositions for the treatment of Alzheimer's Disease included but are not limited to cholinesterase inhibitors (CIs) or inhibitors of NMDA receptor activity. Examples of cholinesterase inhibitors (CIs) include but are not limited to tacrine, donepezil, rivastigmine, and galantamine. An example of an inhibitor of NMDA receptor activity is memantine.

According to a fifth aspect of the invention there is provided a method for the second-line treatment of a neurodegenerative disease in a subject, the method comprising:
(a) assessing the efficacy of a first-line treatment for a neurodegenerative disease in a subject, the method comprising:
(i) preparing a biological sample from the subject for assay by incubating the sample with a protease to form a proteolytic digest of proteins in the sample; and
(ii) assaying the proteolytic digest of proteins of step (i) for the presence of a proteolytic peptide of at least one protein selected from the group of proteins as shown in Table 1, said group consisting of Matrix metalloproteinase-9, Chitinase-3-like protein 1, Protein S100-A8, Protein S100-A9, Neutrophil collagenase (MMP8). Complement C3, Galectin-3-binding protein, Catalase, Extracellular superoxide dismutase, Prosaposin, Beta-hexosaminidase subunit beta, Cathepsin D, Beclin-1, Lysosome-associated membrane glycoprotein 1, Autophagy protein 13, E3 ubiquitin-protein ligase RNF26, E3 ubiquitin-protein ligase TRIM33, FAST kinase domain-containing protein 5, mitochondrial, Apoptosis-inducing factor 1 mitochondrial, Transmembrane protein 126A, Neurofilament light polypeptide, Chromogranin-A, Contactin-1, Neurexin-1-beta, Protein MTSS 1 (Metastasis suppressor YGL-1), Synaptotagmin-3, Apolipoprotein E, Apolipoprotein D, Clusterin, Flotillin-2, Cadherin-2, Neural cell adhesion molecule 2, Amyloid-beta precursor protein, Amyloid-like protein 1, Peptidyl-prolyl cis-trans isomerase FKBP3, Peptidyl-prolyl cis-trans isomerase FKBP4, Kallikrein-6, Neutrophil gelatinase-associated lipocalin, Protein disulfide-isomerase A1 (PDIA1), Limbic system-associated membrane protein (Fragment), Ceruloplasmin, Serotransferrin, Vacuolar protein sorting-associated protein 16 homolog (hVPS16), Peptidyl-prolyl cis-trans isomerase (PPlase; HEL-S-39), Lymphocyte cytosolic protein 2, Lactadherin, 72 kDa type IV collagenase, Transforming growth factor-beta-induced protein ig-h3, Neural proliferation differentiation and control protein 1, Ganglioside GM2 activator,
wherein the presence of said proteolytic peptide is assayed for using mass spectrometry with reference to a corresponding labelled and/or unlabelled reference proteolytic peptide; and
(b) administering a pharmaceutical composition to the subject for the second-line treatment of the neurodegenerative disease.

The first-line treatment of the neurodegenerative disease may be any suitable prescribed treatment regime. The first-line method treatment and the second-line treatment may be the same or different in terms of the pharmaceutical composition administered to the subject, dosage form and/or dosage regime.

Suitable pharmaceutical compositions for the treatment of Parkinson's Disease included, but are not limited to: bromocriptine, cabergoline, lazabemide, levodopa, pergolide, pramipexole, rasagiline, ropinirole, rotigotine and selegiline.

Suitable pharmaceutical compositions for the treatment of Alzheimer's Disease included but are not limited to cholinesterase inhibitors (CIs) or inhibitors of NMDA receptor activity. Examples of cholinesterase inhibitors (CIs) include but are not limited to tacrine, donepezil, rivastigmine, and galantamine. An example of an inhibitor of NMDA receptor activity is memantine.

In addition to the above, the present invention can be commercialised in an assay kit format. Such kits may include a standardised set of consumables shipped to customer sites, alongside instructions on how to perform a test in their laboratories. Alternatively, instructions (standard operating procedure) detailing how to perform the test can be sold separately without consumables.

According to a sixth aspect of the invention, there is provided a kit for assessing the efficacy of a first-line treatment for a neurodegenerative disease in a subject, comprising:
a plurality of sample preparation media for analysis of a sample by mass spectrometry for the presence of a proteolytic peptide of at least one protein selected from the group of proteins as shown in Table 1, said group consisting of Matrix metalloproteinase-9, Chitinase-3-like protein 1, Protein S100-A8, Protein S100-A9, Neutrophil collagenase (MMP8). Complement C3, Galectin-3-binding protein, Catalase, Extracellular superoxide dismutase, Prosaposin, Beta-hexosaminidase subunit beta, Cathepsin D, Beclin-1, Lysosome-associated membrane glycoprotein 1, Autophagy protein 13, E3 ubiquitin-protein ligase RNF26, E3 ubiquitin-protein ligase TRIM33, FAST kinase domain-containing protein 5, mitochondrial, Apoptosis-inducing factor 1 mitochondrial, Transmembrane protein 126A, Neurofilament light polypeptide, Chromogranin-A, Contactin-1, Neurexin-1-beta, Protein MTSS 1 (Metastasis suppressor YGL-1), Synaptotagmin-3, Apolipoprotein E, Apolipoprotein D, Clusterin, Flotillin-2, Cadherin-2, Neural cell adhesion molecule 2, Amyloid-beta precursor protein, Amyloid-like protein 1, Peptidyl-prolyl cis-trans isomerase FKBP3, Peptidyl-prolyl cis-trans isomerase FKBP4, Kallikrein-6, Neutrophil gelatinase-associated lipocalin, Protein disulfide-isomerase A1 (PDIA1), Limbic system-associated membrane protein (Fragment), Ceruloplasmin, Serotransferrin, Vacuolar protein sorting-associated protein 16 homolog (hVPS16), Peptidyl-prolyl cis-trans isomerase (PPlase; HEL-S-39), Lymphocyte cytosolic protein 2, Lactadherin, 72 kDa type IV collagenase, Transforming growth factor-beta-induced protein ig-h3, Neural proliferation differentiation and control protein 1, Ganglioside GM2 activator.

The sample preparation media may comprise a protease. The protease is a serine protease, a cysteine protease, a threonine protease, an aspartic protease, a glutamic protease or a metalloprotease. The protease may be trypsin. Alternatively, the sample preparation media may be suitable for use with a protease.

The sample preparation media may comprise a buffer. The buffer may be suitable for use with a protease. Peptide digestion may take place in the sample preparation media. The skilled person is aware of suitable buffers for proteases.

The sample preparation media may comprise peptide internal standards such as isotopically-labelled peptide internal standards.

The present invention provides is the first comprehensive targeted proteomics biomarker panel for neurodegeneration. It covers all aspects of Parkinson's disease pathology in a single, robust assay. Every molecule in the panel is measured in the most accurate way possible as there is a calibration line and a dedicated internal standard for each analyte. The panel provides absolute concentration of each analyte and is suitable for ongoing clinical testing. Overall, this test provides fundamentally richer and more reliable biomarker data.

Preferred features of the second and subsequent aspects of the invention are as for the first aspect mutatis mutandis.

The invention will now be described by way of reference to the following Examples which are present for the purposes of illustration only and are not to be construed as being limitations on the present invention.

Reference is also made to the following drawings in which:

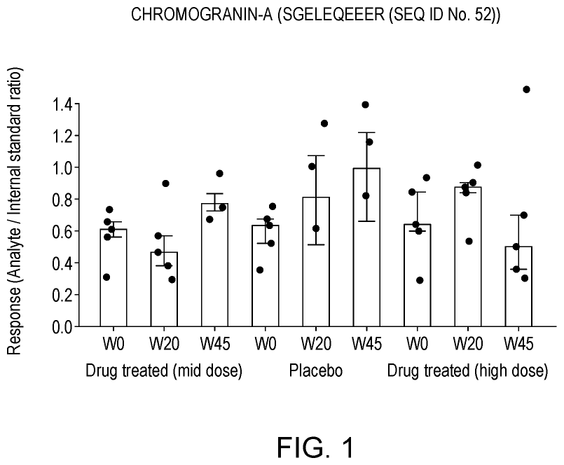

FIG. 1 shows the results of differential protein expression for chromogranin-A in Parkinson's Disease patients. Bars represent medians; error bars represent interquartile range (IQR); dots represent individual samples. W0—baseline, W20—patients after 20 weeks of treatment with drug/placebo, W45—patients after 45 weeks of treatment with drug/placebo

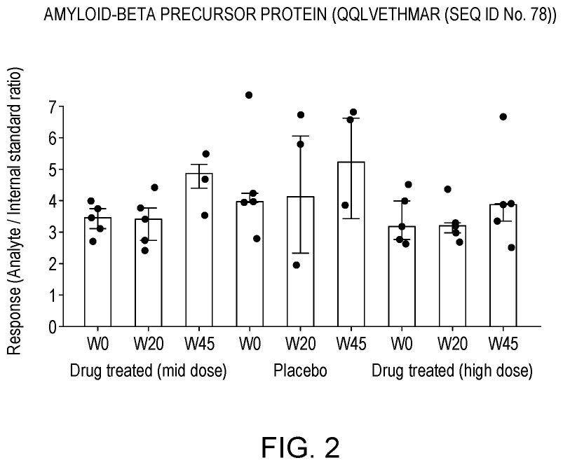

FIG. 2 shows the results of differential protein expression for amyloid-beta precursor protein in Parkinson's Disease patients. Bars represent medians; error bars represent interquartile range (IQR); dots represent individual samples. W0—baseline, W20—patients after 20 weeks of treatment with drug/placebo, W45—patients after 45 weeks of treatment with drug/placebo

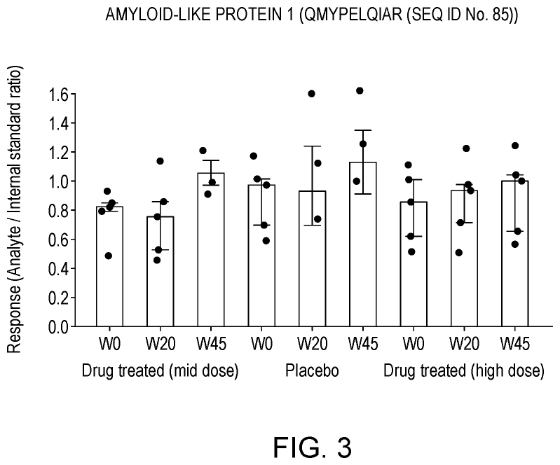

FIG. 3 shows the results of differential protein expression for amyloid-like protein 1 in Parkinson's Disease patients. Bars represent medians; error bars represent interquartile range (IQR); dots represent individual samples. W0—baseline, W20—patients after 20 weeks of treatment with drug/placebo, W45—patients after 45 weeks of treatment with drug/placebo

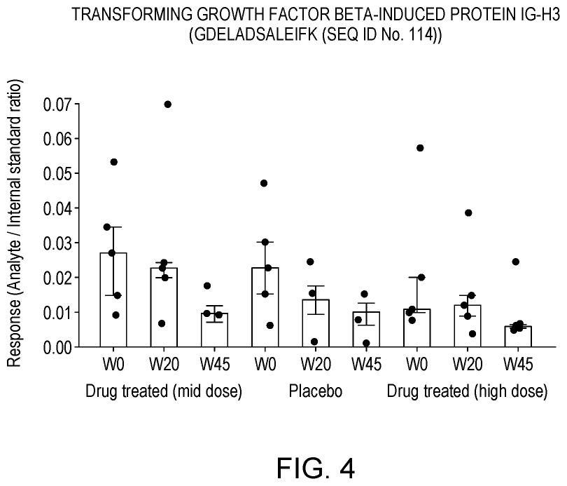

FIG. 4 shows the results of differential protein expression for transforming growth factor-beta-induced protein IG-H3 in Parkinson's Disease patients. Bars represent medians; error bars represent interquartile range (IQR); dots represent individual samples. W0—baseline, W20—patients after 20 weeks of treatment with drug/placebo, W45—patients after 45 weeks of treatment with drug/placebo

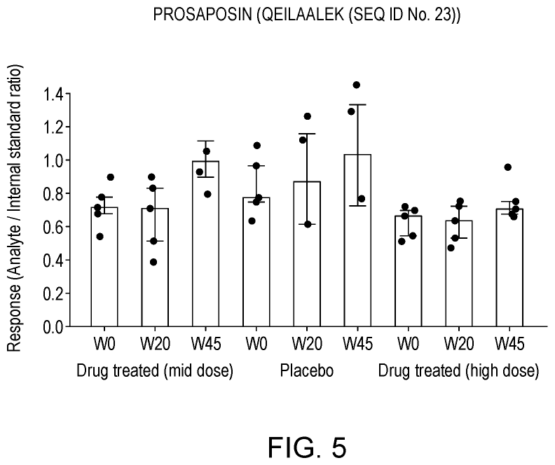

FIG. 5 shows the results of differential protein expression for prosaposin in Parkinson's Disease patients. Bars represent medians; error bars represent interquartile range (IQR); dots represent individual samples. W0—baseline, W20—patients after 20 weeks of treatment with drug/placebo, W45—patients after 45 weeks of treatment with drug/placebo

FIG. 6 shows the summary of differential protein expression for all proteins in the biomarker test. Forward hatching represents fold change increase, backward hatching represents fold change decrease compared to baseline. W0—baseline, W20—patients after 20 weeks of treatment with drug/placebo, W45—patients after 45 weeks of treatment with drug/placebo. FC—fold change.

FIG. 7 shows the summary of differential protein expression for all proteins within a drug treated patient group. Different biomarker trajectories demonstrate that different patients respond to treatment differently and this biomarker test may be used for patient stratification. Forward hatching represents fold change increase, backward hatching represents fold change decrease compared to baseline. W0—baseline, W20—patients after 20 weeks of treatment with drug/placebo, W45—patients after 45 weeks of treatment with drug/placebo. FC—fold change, M0—months.

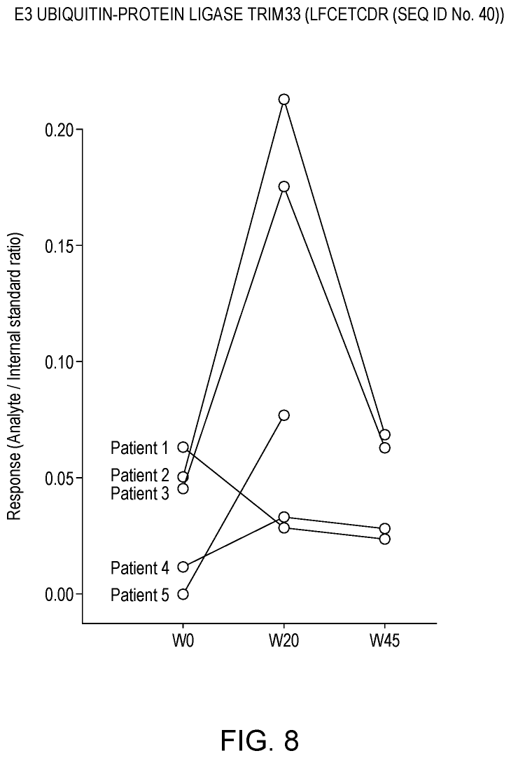

FIG. 8 shows the results of differential protein expression for E3 UBIQUITIN-PROTEIN LIGASE TRIM33 in Parkinson's Disease patients treated with a drug. Different biomarker trajectories demonstrate that different patients respond to treatment differently and this biomarker test may be used for patient stratification. Dots represent individual samples. W0—baseline, W20—patients after 20 weeks of treatment with drug, W45—patients after 45 weeks of treatment with drug.

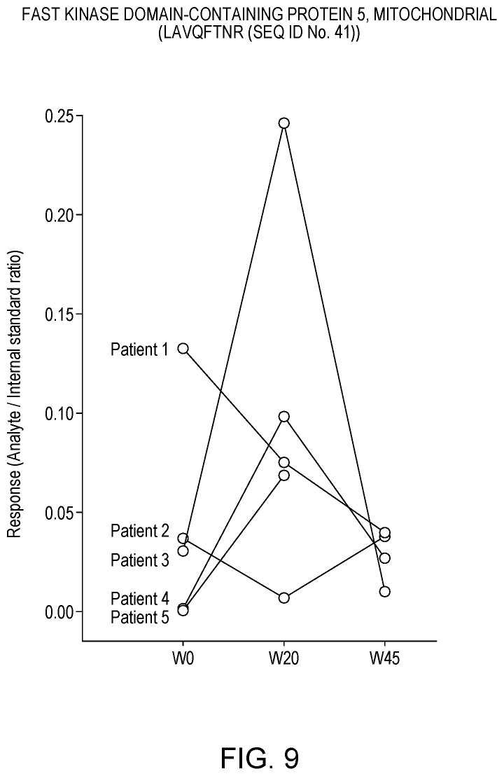

FIG. 9 shows the results of differential protein expression for FAST KINASE DOMAIN-CONTAINING PROTEIN 5, MITOCHONDRIAL in Parkinson's Disease patients treated with a drug. Different biomarker trajectories demonstrate that different patients respond to treatment differently and this biomarker test may be used for patient stratification. Dots represent individual samples. W0—baseline, W20—patients after 20 weeks of treatment with drug, W45—patients after 45 weeks of treatment with drug.

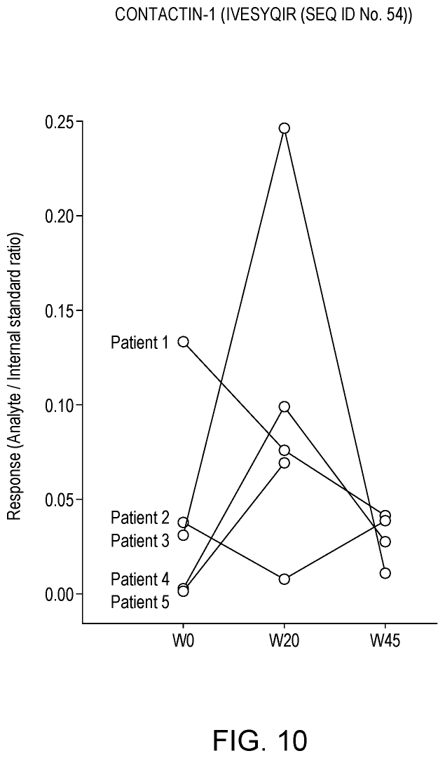

FIG. 10 shows the results of differential protein expression for CONTACTIN-1 in Parkinson's Disease patients treated with a drug. Different biomarker trajectories demonstrate that different patients respond to treatment differently and this biomarker test may be used for patient stratification, Dots represent individual samples. W0—baseline, W20—patients after 20 weeks of treatment with drug, W45—patients after 45 weeks of treatment with drug.

Protein biomarker names indicated in FIGS. 6 and 7 are referred to as “Biomarker 1” to “Biomarker 48”, which correspond to the 48 of the 50 proteins of Table 1; the two proteins included in Table 1 but omitted from FIGS. 6 and 7 are Protein MTSS 1 (Metastasis suppressor YGL-1) and Lymphocyte cytosolic protein 2.

The method may comprise detecting an increase and/or a decrease of any one or more proteolytic peptide wherein FIG. 6 and/or FIG. 7 indicates an increase and/or a decrease for the corresponding biomarker. The increase and/or decrease may be detected in a sample obtained at any time point where an increase and/or decrease is indicated in FIG. 6 (for example week 20 or week 45) and/or FIG. 7 (for example, month 6 or month 12). With reference to FIG. 6, the biomarker may be a biomarker that decreases in placebo at week 20 and/or week 45. With reference to FIG. 6, the biomarker may be a biomarker that increases in one or more drug-treated group at week 20 and/or week 45. With reference to FIG. 6, the biomarker may be a biomarker that decreases in placebo at week 20 and/or week 45 and increases in one or more drug-treated group at week 20 and/or week 45. For example, the biomarker may be biomarker 23 which decreases in placebo at week 20 and increases in both drug-treated groups at week 20. For example, the biomarker may be biomarker 33 which decreases in placebo at week 20 and increases in both drug-treated groups at week 20. For example, the biomarker may be biomarker 3 which decreases in placebo at week 20 and increases in the mid-dose drug-treated group at week 20. For example, the biomarker may be biomarker 41 which decreases in placebo at week 20 and week 45 and increases in the mid-dose drug-treated group at week 20 and increases in the high-dose drug-treated group at week 20 and week 45. The increase and/or decrease may be a fold change relative to placebo for any biomarker in FIG. 6. The biomarker may be any biomarker where FIG. 7 indicates a difference between any two patients at any time point, for example at 6 months and/or at 12 months. For example, the biomarker may be biomarker 48 which increases in patient 3 months at 6 months and at 12 months and decreases in patients 2 and 4 at 3 months and 6 months. Biomarker 48 is therefore an example of a biomarker which may be used to stratify patients by treatment response according to the relative concentration of a peptide measured in the sample. The biomarker may be any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 of the following proteins:
Matrix metalloproteinase-9
Chitinase-3-like protein 1
Protein S100-A8
Protein S100-A9
Neutrophil collagenase (MMP8)
Complement C3
Galectin-3-binding protein
Catalase
Extracellular superoxide dismutase
Prosaposin
Beta-hexosaminidase subunit beta
Cathepsin D
Beclin-1
Lysosome-associated membrane glycoprotein 1
Autophagy protein 13
E3 ubiquitin-protein ligase RNF26
E3 ubiquitin-protein ligase TRIM33
FAST kinase domain-containing protein 5, mitochondrial
Apoptosis-inducing factor 1, mitochondrial
Transmembrane protein 126A
Neurofilament light polypeptide
Chromogranin-A
Contactin-1
Neurexin-1-beta
Synaptotagmin-3
Apolipoprotein E
Apolipoprotein D
Clusterin
Flotillin-2
Cadherin-2
Neural cell adhesion molecule 2
Amyloid-beta precursor protein
Amyloid-like protein 1
Peptidyl-prolyl cis-trans isomerase FKBP3
Peptidyl-prolyl cis-trans isomerase FKBP4
Kallikrein-6
Neutrophil gelatinase-associated lipocalin
Protein disulfide-isomerase A1 (PDIA1)
Limbic system-associated membrane protein (Fragment)
Ceruloplasmin
Serotransferrin
Vacuolar protein sorting-associated protein 16 homolog (hVPS16)
Peptidyl-prolyl cis-trans isomerase (PPlase; HEL-S-39)
Lactadherin
72 kDa type IV collagenase
Transforming growth factor-beta-induced protein ig-h3
Neural proliferation differentiation and control protein 1 and
Ganglioside GM2 activator.

Therefore, the at least one protein used in the method or kit of the invention may be any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 of the 48 proteins listed above. Preferably, the at least one protein may be all 48 of the proteins listed above. Accordingly, the method may comprise assaying the proteolytic digest of proteins of step (i) for the presence of a proteolytic peptide of all 48 of the proteins listed above.

Further advantages of the invention include:
Showing it is possible to develop a highly multiplexed biomarker assay that covers all aspects of Parkinson's Disease molecular pathology in a single test. Table 1 details these disease processes, proteins selected and their corresponding tryptic peptides. The inventors believe this is the world's first comprehensive targeted proteomics biomarker panel for Parkinson's Disease/neurodegeneration.
Showing it is possible to apply this test to stratify patient population and to assess drug treatment effects (FIGS. 6 & 7).
Showing how to set up this biomarker test on a specific analytical instrument which a skilled person in the field will be able to do by referring to Tables 1 & 2 and application text. Table 2 discloses specific instruments settings to the level of detail where these can be directly copied to set up this biomarker test.
Revealing proteins that were previously not associated with Parkinson's Disease, and that these proteins are a part of the method to stratify patient populations in Parkinson's Disease and to assess drug treatment effects.
Showing that it is possible to establish this comprehensive panel of biomarkers on specific analytical instruments which provide more accurate and robust analytical measurements for every single molecule in the panel compared to other analytical technologies; and that this assay can be validated to existing regulatory standards in clinical settings.
Overall, this biomarker test provides fundamentally richer and more reliable biomarker data compared to other technologies in the field.

### EXAMPLE 1: PROTEOMIC ANALYSIS OF CSF SAMPLES FROM PARKINSON'S DISEASE (PD) PATIENTS UNDERGOING TREATMENT IN CLINICAL TRIAL

### Sample Collection

CSF samples were collected from Parkinson's Disease patients by lumbar puncture in a clinical trial at baseline, 20 weeks and 45 weeks after treatment with placebo or a PD drug in development.

### Sample Preparation

Equal volumes of CSF were freeze-dried and isotopically-labelled peptide internal standards were added prior to protein digestion. CSF proteins were enzymatically digested into peptides using Trypsin, with prior reduction and alkylation. After digestion, samples were purified using a C18-based solid phase extraction microplate before injection into LC-MS/MS system.

### LC-MS/MS Set Up

The tryptic peptides were then quantified on a liquid chromatography—tandem mass spectrometry system (LC-MS/MS), operated in timed multiple reaction monitoring mode (MRM). A Thermo Scientific TSQ Altis triple quadrupole mass spectrometer was used for quantification. At least 2 unique tryptic peptides were quantified for each protein biomarker, using corresponding internal standards as shown in Table 1 and peptide calibration lines in assay buffer and CSF. A minimum of 2 transitions were used for the confident identification of each peptide as detailed in Table 2 and Table 3. Each patient sample was injected onto a Kinetex reversed phase C18-based liquid chromatography column and peptides separated with water and acetonitrile gradient at 0.5 ml/min flow rate. The samples were run blinded and in a random order. Chromatograms were analyzed using Xcalibur v4.1 software and absolute concentration was obtained from standard curves, constructed with peptides shown in Table 1. A standard curve of each peptide was analyzed at the start and the end of the run for quantitation and performance standardization.

### Statistical Analysis/Results Interpretation

A linear regression model was fitted through the assay buffer calibration line dataset to obtain absolute peptide concentration. The calibration line was plotted on a scatter plot with logarithmic axes (log-log) and 1/x² weighting was applied. The lower limits of quantification, intra-day and inter-day coefficient of variation (% CV) were also established for each peptide at multiple concentrations. Absolute peptide concentration was then compared across the sample set. Week 45 and Week 20 results were normalised to baseline and results expressed as fold change from baseline. 1.5-fold peptide concentration increase or 0.67-fold concentration decrease from baseline in drug treated groups whilst having no or lesser corresponding peptide alteration in the placebo group were considered to indicate drug treatment efficacy.

### EXAMPLE 2: STRATIFICATION OF PATIENTS

To stratify patient groups after drug treatment, biomarker (i.e. peptide) trajectories within a drug treated patient group were compared. All patients with biomarker signal increase were stratified into subgroup 1 whereas all patients with biomarker signal decrease or not significantly changing biomarker signal were classified into subgroup 2.

Reference is also made in the present application to the following Table:

Table 1 shows 50 proteins differentially expressed in Parkinson's Disease patients and corresponding 118 proteolytic peptide sequences with corresponding heavy isotope-labelled peptide internal standard sequences.

| Disease process | | | |
|---|---|---|---|
| relevant to | | | |
| Parkinson's | | Corresponding | Corresponding |
| disease | Protein name | proteolytic peptide sequence | heavy isotope-labelled peptide internal standard sequence |
| None (analytical | Haemoglobin | VNVDEVGGEALGR (SEQ ID No. 1) | ALWGKVNVDEVGGEALGR(U-13C6,15N4) (SEQ ID No. 119) |
| control) | subunit beta | | |
| Inflammation | Matrix | AFALWSAVTPLTFTR (SEQ ID No. 2) | |
| | metalloproteinase- | | |
| | 9 | QLSLPETGELDSATLK (SEQ ID No. 3) | LLLQKQLSLPETGELDSATLK(U-13C6,15N2) (SEQ ID No. 120) |
| | Chitinase-3-like | LVMGIPTFGR (SEQ ID No. 4) | |
| | protein 1 | TLLSVGGWNFGSQR (SEQ ID No. 5) | NPNLKTLLSVGGWNFGSQR(U-13C6,15N4) (SEQ ID No. 121) |
| | | FSNTDYAVGYMLR (SEQ ID No. 6) | FSNTDYAVGYMLR(U-13C6,15N4)LGAPA (SEQ ID No. 122) |
| | Protein S100-A8 | ALNSIIDVYHK (SEQ ID No. 7) | ALNSIIDVYHK(U-13C6,15N2)YSLIK (SEQ ID No. 123) |
| | | GNFHAVYR (SEQ ID No. 8) | YSLIKGNFHAVYR(U-13C6,15N4) (SEQ ID No. 124) |
| | Protein S100-A9 | VIEHIMEDLDTNADK (SEQ ID No. 9) | VIEHIMEDLDTNADK(U-13C6,15N2)QLSFE (SEQ ID No. 125) |
| | | LTWASHEK (SEQ ID No. 10) | LTWASHEK(U-13C6,15N2)MHEGD (SEQ ID No. 126) |
| | Neutrophil | YYAFDLIAQR (SEQ ID No. 11) | FSGPRYYAFDLIAQR(U-13C6,15N4) (SEQ ID No. 127) |
| | collagenase | | |
| | (MMP8) | VDAVFQQEHFFHVFSGPR (SEQ ID No. 12) | |
| | Complement C3 | VLLDGVQNPR (SEQ ID No. 13) | |
| | | NTLIIYLDK (SEQ ID No. 14) | NTLIIYLDK(U-13C6,15N2)VSHSE (SEQ ID No. 128) |
| | Galectin-3-binding | ASHEEVEGLVEK (SEQ ID No. 15) | |
| | protein | SQLVYQSR (SEQ ID No. 16) | SQLVYQSR(U-13C6,15N4)RGPLV (SEQ ID No. 129) |
| | | IYTSPTWSAFVTDSSWSAR (SEQ ID No. 17) | |
| Oxidative stress | Catalase | FSTVAGESGSADTVR (SEQ ID No. 18) | |
| | Extracellular | GAGAFGYFEVTHDITK (SEQ ID No. 19) | VVHAKGAGAFGYFEVTHDITK(U-13C6,15N2) (SEQ ID No. 130) |
| | superoxide | AGLAASLAGPHSIVGR (SEQ ID No. 20) | AGLAASLAGPHSIVGR(U-13C6,15N4)AVVVH (SEQ ID No. 131) |
| | dismutase | AVVVHAGEDDLGR (SEQ ID No. 21) | |
| Autophagy/ | Prosaposin | EILDAFDK (SEQ ID No. 22) | |
| Lysosomal | | QEILAALEK (SEQ ID No. 23) | QEILAALEK(U-13C6,15N2)GCSFL (SEQ ID No. 132) |
| | Beta- | VEPLDFGGTQK (SEQ ID No. 24) | YYKVEPLDFGGTQK(U-13C6,15N2) (SEQ ID No. 133) |
| | hexosaminidase | | |
| | subunit beta | VLDIIATINK (SEQ ID No. 25) | |
| | Cathepsin D | LSPEDYTLK (SEQ ID No. 26) | LSPEDYTLK(U-13C6,15N2)VSQAG (SEQ ID No. 134) |
| | | VSTLPAITLK (SEQ ID No. 27) | |
| | Beclin-1 | WGLAWVSSQFYNK (SEQ ID No. 28) | LTNLKWGLAWVSSQFYNK(U-13C6,15N4) (SEQ ID No. 135) |
| | | TSNNSTMQVSFVCQR (SEQ ID No. 29) | |
| | | ELALEEER (SEQ ID No. 30) | |
| | Lysosome- | ENTSDPSLVIAFGR (SEQ ID No. 31) | |
| | associated | | |
| | membrane | TVESITDIR (SEQ ID No. 32) | |
| | glycoprotein 1 | ALQATVGNSYK (SEQ ID No. 33) | ALQATVGNSYK(U-13C6,15N2)CNAEE (SEQ ID No. 136) |
| | Autophagy protein | DDILPMDLGTFYR (SEQ ID No. 34) | DDILPMDLGTFYR(U-13C6,15N4)EFQNP (SEQ ID No. 137) |
| | 13 | TVQVIVQAR (SEQ ID No. 35) | |
| Ubiquitin | E3 ubiquitin- | LLGHLASHGALR (SEQ ID No. 36) | |
| Proteasome | protein ligase | QDTLPEAGR (SEQ ID No. 37) | QDTLPEAGR(U-13C6,15N4)RSEAE (SEQ ID No. 138) |
| System (UPS) | RNF26 | SLQLASWPNR (SEQ ID No. 38) | |
| | E3 ubiquitin- | QIDLVDNYFVK (SEQ ID No. 39) | QIDLVDNYFVK(U-13C6,15N2)DTSEA (SEQ ID No. 139) |
| | protein ligase | | |
| | TRIM33 | LFCETCDR (SEQ ID No. 40) | |
| Mitochondrial | FAST kinase | LAVQFTNR (SEQ ID No. 41) | LAVQFTNR(U-13C6,15N4)NQYCY (SEQ ID No. 140) |
| | domain-containing | ASTLQLGSPR (SEQ ID No. 42) | |
| | protein 5, | | |
| | mitochondrial | | |
| | Apoptosis- | GVIFYLR (SEQ ID No. 43) | EDYGKGVIFYLR(U-13C6,15N4) (SEQ ID No. 141) |
| | inducing factor 1, | ELWFSDDPNVTK (SEQ ID No. 44) | |
| | mitochondrial | | |
| | Transmembrane | YQSALLPHK (SEQ ID No. 45) | |
| | protein 126A | ENITIVDISR (SEQ ID No. 46) | ENITIVDISR(U-13C6,15N4)KINQL (SEQ ID No. 142) |
| Axonal/neuronal | Neurofilament | YEEEVLSR (SEQ ID No. 47) | |
| degeneration | light polypeptide | IDSLMDEISFLK (SEQ ID No. 48) | IDSLMDEISFLK(U-13C6,15N2)KVHEE (SEQ ID No. 143) |
| | | VLEAELLVLR (SEQ ID No. 49) | EQQNKVLEAELLVLR(U-13C6,15N4) (SEQ ID No. 144) |
| | | FTVLTESAAK (SEQ ID No. 50) | FTVLTESAAK(U-13C6,15N2)NTDAV (SEQ ID No. 145) |
| Synaptic | Chromogranin-A | YPGPQAEGDSEGLSQGLVDR (SEQ ID No. 51) | YPGPQAEGDSEGLSQGLVDR(U-13C6,15N4)EKGLS (SEQ ID No. 146) |
| degeneration | | SGELEQEEER (SEQ ID No. 52) | |
| | Contactin-1 | FIPLIPIPER (SEQ ID No. 53) | SVFSKFIPLIPIPER(U-13C6,15N4) (SEQ ID No. 147) |
| | | IVESYQIR (SEQ ID No. 54) | |
| | Neurexin-1-beta | SDLYIGGVAK (SEQ ID No. 55) | NLDLKSDLYIGGVAK(U-13C6,15N2) (SEQ ID No. 148) |
| | | ITTQITAGAR (SEQ ID No. 56) | |
| | Protein MTSS 1 | TTVVAAAAFLDAFQK (SEQ ID No. 57) | TTVVAAAAFLDAFQK(U-13C6,15N2)VADMA (SEQ ID No. 149) |
| | (Metastasis | | |
| | suppressor YGL-1) | SLTMDPHK (SEQ ID No. 58) | |
| | Synaptotagmin-3 | YLYGSDQLVVR (SEQ ID No. 59) | |
| | | ILQALDLPAK (SEQ ID No. 60) | |
| | | KPVEHWHQLVEVSR (SEQ ID No. 61) | LANPRKPVEHWHQLVEVSR(U-13C6,15N2) (SEQ ID No. 150) |
| | | GPTPTPDK (SEQ ID No. 62) | |
| | | HPC(Carbamidomethyl)TLGPFIHATNALHVR | |
| | | (SEQ ID No. 63) | |
| Lipoprotein | Apolipoprotein E | AATVGSLAGQPLQER (SEQ ID No. 64) | AATVGSAGQPLQER(U-13C6,15N4)AQAWG (SEQ ID No. 151) |
| metabolism | | GEVQAMLGQSTEELR (SEQ ID No. 65) | |
| | | LGPLVEQGR (SEQ ID No. 66) | LGPLVEQGR(U-13C6,15N4)VRAAT (SEQ ID No. 152) |
| | Apolipoprotein D | IPTTFENGR (SEQ ID No. 67) | IPTTFENGR(U-13C6,15N4)CIQAN (SEQ ID No. 153) |
| | | VLNQELR (SEQ ID No. 68) | |
| | Clusterin | VTTVASHTSDSDVPSGVTEVVVK (SEQ ID No. | VTTVASHTSDSDVPSGVTEVVVK(U-13C6,15N2)LFDSD (SEQ ID No. |
| | | 69) | 154) |
| | | ASSIIDELFODR (SEQ ID No. 70) | |
| Enhanced | Flotillin-2 | NVVLQTLEGHLR (SEQ ID No. 71) | |
| release of | | EGHLRSILGTLTVEQIYQDRDQFAK (SEQ ID | EGHLRSILGTLTVEQIYQDR(U-13C6,15N4)DQFAK (SEQ ID No. 155) |
| exosomes | | No. 72) | |
| | | TAEAQLAYELQGAR (SEQ ID No. 73) | |
| Endothelial | Cadherin-2 | GPFPQELVR (SEQ ID No. 74) | |
| dysfunction | | DVHEGQPLLNVK (SEQ ID No. 75) | DVHEGQPLLNVK(U-13C6,15N4)FSNCN (SEQ ID No. 156) |
| | Neural cell | EVVSPQEFK (SEQ ID No. 76) | EVVSPQEFK(U-13C6,15N2)QGEDA (SEQ ID No. 157) |
| | adhesion molecule | | |
| | 2 | ALLQVTISLSK (SEQ ID No. 77) | |
| Amyloid | Amyloid-beta | QQLVETHMAR (SEQ ID No. 78) | |
| Processing | precursor protein | GAIIGLMVGGVV (SEQ ID No. 79) | VGSNKGAIIGLMVGGV(U-13C5,15N)V(U-13C5,15N) (SEQ ID No. 158) |
| | | GAIIGLMVGGVVIA (SEQ ID No. 80) | VGSNKGAIIGLMVGGV(U-13C5,15N)V(U-13C5,15N)IA (SEQ ID No. |
| | | | 159) |
| | | LVFFAEDVGSNK (SEQ ID No. 81) | LVFFAEDVGSNK(U-13C6,15N2)GAIIG (SEQ ID No. 160) |
| | | WYFDVTEGK (SEQ ID No. 82) | |
| | | DDTPMTLPK (SEQ ID No. 83) | DDTPMTLPK(U-13C6,15N2)GSTEQ (SEQ ID No. 161) |
| | Amyloid-like | HGYENPTYR (SEQ ID No. 84) | |
| | protein 1 | QMYPELQIAR (SEQ ID No. 85) | QMYPELQIAR(U-13C6,15N4)VEQAT (SEQ ID No. 162) |
| Other proteins | Peptidyl-prolyl cis- | AWTVEQLR (SEQ ID No. 86) | AWTVEQLR(U-13C6,15N4)SEQLP (SEQ ID No. 163) |
| relevant to | trans isomerase | | |
| Parkinson's | FKBP3 | SEETLDEGPPK (SEQ ID No. 87) | |
| disease or | Peptidyl-prolyl cis- | ESWEMNSEEK (SEQ ID No. 88) | ESWEMNSEEK(U-13C6,15N2)LEQST (SEQ ID No. 164) |
| therapeutics for | trans isomerase | | |
| Parkinson's | FKBP4 | ALELDSNNEK (SEQ ID No. 89) | |
| disease | Kallikrein-6 | LSELIQPLPLER (SEQ ID No. 90) | LSELIQPLPLER(U-13C6,15N4)DCSAN (SEQ ID No. 165) |
| | | YTNWIQK (SEQ ID No. 91) | |
| | Neutrophil | MYATIYELK (SEQ ID No. 92) | KDPQKMYATIYELK(U-13C6,15N2) (SEQ ID No. 166) |
| | gelatinase- | | |
| | associated | VPLQQNFQDNQFQGK (SEQ ID No. 93) | |
| | lipocalin | | |
| | Protein disulfide- | MDSTANEVEAVK (SEQ ID No. 94) | MDSTANEVEAVK(U-13C6,15N2)VHSFP (SEQ ID No. 167) |
| | isomerase A1 | | |
| | (PDIA1) | ALAPEYAK (SEQ ID No. 95) | |
| | Limbic system- | VTVNYPPTITESK (SEQ ID No. 96) | VTVNYPPTITESK(U-13C6,15N2)SNEAT (SEQ ID No. 168) |
| | associated | | |
| | membrane protein | SGIIFAGHDK (SEQ ID No. 97) | |
| | (Fragment) | | |
| | Ceruloplasmin | MYYSAVEPTK (SEQ ID No. 98) | MYYSAVEPTK(U-13C6,15N2)DIFTG (SEQ ID No. 169) |
| | | IYHSHIDAPK (SEQ ID No. 99) | |
| | Serotransferrin | DSAHGFLK (SEQ ID No. 100) | DLLFKDSAHGFLK(U-13C6,15N2) (SEQ ID No. 170) |
| | | DGAGDVAFVK (SEQ ID No. 101) | |
| | Vacuolar protein | LGDTPGVSYSDIAAR (SEQ ID No. 102) | |
| | sorting-associated | | |
| | protein 16 | ALLLVGDVAQAADVAIEHR | ALLLVGDVAQAADVAIEHR(U-13C6,15N4)NEAEL (SEQ ID No. 171) |
| | homolog (hVPS16) | (SEQ ID No. 103) | |
| | Peptidyl-prolyl cis- | TVDNFVALATGEK (SEQ ID No. 104) | TVDNFVALATGEK(U-13C6,15N2)GFGYK (SEQ ID No. 172) |
| | trans isomerase | | |
| | (PPlase; HEL-S-39) | VYFDLR (SEQ ID No. 105) | |
| | Lymphocyte | FLNLTENDIQK (SEQ ID No. 106) | |
| | cytosolic protein 2 | LPAPSIDR (SEQ ID No. 107) | |
| | | GKEDFLSVSDIIDYFR (SEQ ID No. 108) | GKEDFLSVSDIIDYFR(U-13C6,15N4)KMPLL (SEQ ID No. 173) |
| | Lactadherin | VTFLGLQHWVPELAR (SEQ ID No. 109) | ASSVRVTFLGLQHWVPELAR(U-13C6,15N4) (SEQ ID No. 174) |
| | | LASHEYLK (SEQ ID No. 110) | |
| | 72 kDa type IV | AFQVWSDVTPLR (SEQ ID No. 111) | DAFARAFQVWSDVTPLR(U-13C6,15N4) (SEQ ID No. 175) |
| | collagenase | FFGLPQTGDLDQNTIETMR (SEQ ID No. 112) | |
| | Transforming | LTLLAPLNSVFK (SEQ ID No. 113) | SGSERLTLLAPLNSVFK(U-13C6,15N2) (SEQ ID No. 176) |
| | growth factor- | | |
| | beta-induced | GDELADSALEIFK (SEQ ID No. 114) | |
| | protein ig-h3 | | |
| | Neural | LPEPATLGFSAR (SEQ ID No. 115) | |
| | proliferation | | |
| | differentiation and | NPLFDHAALSAPLPAPSSPPALP (SEQ ID No. | EMEVRNPLFDHAALSAPLPAPSSPPAL(U-13C6,15N)P(U-13C5,15N) |
| | control protein 1 | 116) | (SEQ ID No. 177) |
| | | SEFVVPDLELPSWLTTGNYR (SEQ ID No. 117) | |
| | Ganglioside GM2 | EGTYSLPK (SEQ ID No. 118) | EGTYSLPK(U-13C6,15N2)SEFVV (SEQ ID No. 178) |
| | activator | | |

Table 2 shows experimentally optimised targeted LC-MS/MS conditions to monitor native peptides listed in Table 1. The data in Table 2 is based on ThermoScientific triple quadruple LC-MS/MS platform TSQ Altis but is equally transferable to platforms manufactured by other companies. In Table 2, the following abbreviations are used: RF—Radio Frequency.

TABLE 2

| | Precursor | Product | Collision | | |
|---|---|---|---|---|---|
| Proteolytic peptide sequence | (Q1) m/z | (Q3) m/z | Energy (V) | RF Lens (V) | RT (mins) |
| AFQVWSDVTPLR (SEQ ID No. | 709.99 | 973.46 | 22 | 82 | 6.5 |
| 111) | 709.99 | 385.22 | 18 | 82 | 6.5 |
| | 709.99 | 787.38 | 22 | 82 | 6.5 |
| FFGLPQTGDLDQNTIETMR (SEQ | 1092.12 | 859.96 | 34 | 142 | 7.2 |
| ID No. 112) | 1092.12 | 352.16 | 43 | 142 | 7.2 |
| | 1092.12 | 992.42 | 45 | 142 | 7.2 |
| QMYPELQIAR (SEQ ID No. 85) | 624.93 | 826.46 | 21 | 77 | 5.3 |
| | 624.93 | 413.79 | 18 | 77 | 5.3 |
| | 624.93 | 989.54 | 21 | 77 | 5.3 |
| DDTPMTLPK (SEQ ID No. 83) | 509.34 | 686.38 | 14 | 54 | 4.3 |
| | 509.34 | 244.14 | 24 | 54 | 4.3 |
| | 509.34 | 332.13 | 12 | 54 | 4.3 |
| HGYENPTYR (SEQ ID No. 84) | 568.82 | 999.38 | 23 | 117 | 2.5 |
| | 568.82 | 942.38 | 22 | 117 | 2.5 |
| | 568.82 | 536.28 | 24 | 117 | 2.5 |
| IPTTFENGR (SEQ ID No. 67) | 517.85 | 461.29 | 17 | 77 | 3.7 |
| | 517.85 | 824.38 | 20 | 77 | 3.7 |
| VLNQELR (SEQ ID No. 68) | 436.36 | 659.36 | 14 | 56 | 3.2 |
| | 436.36 | 213.14 | 13 | 56 | 3.2 |
| | 436.36 | 772.39 | 16 | 56 | 3.2 |
| AATVGSLAGQPLQER (SEQ ID | 749.47 | 642.38 | 22 | 95 | 4.5 |
| No. 64) | 749.47 | 827.38 | 24 | 95 | 4.5 |
| GEVQAMLGQSTEELR (SEQ ID | 824.50 | 919.46 | 27 | 102 | 5.5 |
| No. 65) | 824.50 | 1032.49 | 28 | 102 | 5.5 |
| | 824.50 | 1163.54 | 29 | 102 | 5.5 |
| LGPLVEQGR (SEQ ID No. 66) | 484.87 | 588.29 | 20 | 58 | 4.0 |
| | 484.87 | 489.21 | 17 | 58 | 4.0 |
| GVIFYLR (SEQ ID No. 43) | 867.41 | 270.21 | 47 | 168 | 6.1 |
| | 867.41 | 311.13 | 47 | 168 | 6.1 |
| | 867.41 | 434.21 | 41 | 168 | 6.1 |
| ELWFSDDPNVTK (SEQ ID No. | 725.94 | 875.38 | 22 | 85 | 5.9 |
| 44) | 725.94 | 558.29 | 28 | 85 | 5.9 |
| | 725.94 | 1022.46 | 21 | 85 | 5.9 |
| LVFFAEDVGSNK (SEQ ID No. 81) | 663.44 | 213.21 | 21 | 80 | 5.9 |
| | 663.44 | 1113.46 | 19 | 80 | 5.9 |
| | 663.44 | 819.36 | 22 | 80 | 5.9 |
| WYFDVTEGK (SEQ ID No. 82) | 572.84 | 795.38 | 17 | 74 | 5.8 |
| | 572.84 | 350.13 | 16 | 74 | 5.8 |
| GAIIGLMVGGVV (SEQ ID No. | 543.43 | 484.88 | 10 | 59 | 6.8 |
| 79) | 543.43 | 731.38 | 18 | 59 | 6.8 |
| | 543.43 | 242.13 | 19 | 59 | 6.8 |
| GAIIGLMVGGVVIA (SEQ ID No. | 635.48 | 525.29 | 10 | 52 | |
| 80) | 635.48 | 656.38 | 10 | 52 | |
| | 635.48 | 203.14 | 15 | 52 | |
| QQLVETHMAR (SEQ ID No. 78) | 404.97 | 398.96 | 11 | 47 | 3.1 |
| | 404.97 | 372.78 | 11 | 47 | 3.1 |
| | 404.97 | 478.88 | 11 | 47 | 3.1 |
| DDILPMDLGTFYR (SEQ ID No. | 778.43 | 1099.47 | 21 | 83 | 7.4 |
| 34) | 778.43 | 344.13 | 22 | 83 | 7.4 |
| | 778.43 | 457.21 | 17 | 83 | 7.4 |
| | 778.43 | 550.36 | 21 | 83 | 7.4 |
| TVQVIVQAR (SEQ ID No. 35) | 507.41 | 813.46 | 16 | 57 | 3.9 |
| | 507.41 | 586.38 | 17 | 57 | 3.9 |
| | 507.41 | 473.29 | 16 | 57 | 3.9 |
| WGLAWVSSQFYNK (SEQ ID No. | 793.47 | 244.13 | 35 | 105 | 7.3 |
| 28) | 793.47 | 873.38 | 25 | 105 | 7.3 |
| | 793.47 | 972.46 | 25 | 105 | 7.3 |
| TSNNSTMQVSFVCQR (SEQ ID | 879.97 | 796.38 | 30 | 119 | 4.7 |
| No. 29) | 879.97 | 463.18 | 29 | 119 | 4.7 |
| | 879.97 | 1023.46 | 31 | 119 | 4.7 |
| ELALEEER (SEQ ID No. 30) | 494.82 | 562.21 | 15 | 56 | 3.9 |
| | 494.82 | 746.36 | 16 | 56 | 3.9 |
| | 494.82 | 433.21 | 14 | 56 | 3.9 |
| GPFPQELVR (SEQ ID No. 74) | 521.88 | 741.38 | 18 | 70 | 5.3 |
| | 521.88 | 371.29 | 18 | 70 | 5.3 |
| | 521.88 | 302.20 | 16 | 70 | 5.3 |
| | 521.88 | 444.86 | 15 | 70 | 5.3 |
| FSTVAGESGSADTVR (SEQ ID No. | 742.53 | 978.49 | 24 | 130 | 3.6 |
| 18) | 742.53 | 1049.54 | 24 | 130 | 3.6 |
| | 742.53 | 792.45 | 23 | 130 | 3.6 |
| LSPEDYTLK (SEQ ID No. 26) | 533.39 | 433.29 | 14 | 69 | 4.5 |
| | 533.39 | 865.46 | 15 | 69 | 4.5 |
| VSTLPAITLK (SEQ ID No. 27) | 1042.70 | 642.46 | 43 | 186 | 5.5 |
| | 1042.70 | 365.22 | 52 | 186 | 5.5 |
| | 1042.70 | 361.17 | 47 | 186 | 5.5 |
| MYYSAVEPTK (SEQ ID No. 98) | 594.91 | 894.46 | 17 | 79 | 4.0 |
| | 594.91 | 345.21 | 25 | 79 | 4.0 |
| | 594.91 | 731.45 | 18 | 79 | 4.0 |
| IYHSHIDAPK (SEQ ID No. 99) | 394.36 | 452.85 | 12 | 50 | 2.5 |
| | 394.36 | 534.45 | 12 | 50 | 2.5 |
| | 394.36 | 244.21 | 17 | 50 | 2.5 |
| TLLSVGGWNFGSQR (SEQ ID No. | 761.56 | 1008.46 | 24 | 120 | 6.7 |
| 5) | 761.56 | 1194.55 | 25 | 120 | 6.7 |
| | 761.56 | 951.46 | 24 | 120 | 6.7 |
| LVMGIPTFGR (SEQ ID No. 4) | 545.94 | 577.30 | 16 | 61 | 6.4 |
| | 545.94 | 878.46 | 16 | 61 | 6.4 |
| | 545.94 | 213.14 | 16 | 61 | 6.4 |
| FSNTDYAVGYMLR (SEQ ID No. | 769.02 | 639.38 | 23 | 118 | 6.0 |
| 6) | 769.02 | 809.46 | 26 | 118 | 6.0 |
| | 769.02 | 972.47 | 25 | 118 | 6.0 |
| SGELEQEEER (SEQ ID No. 52) | 603.38 | 274.13 | 21 | 81 | 2.5 |
| | 603.38 | 819.38 | 21 | 81 | 2.5 |
| | 603.38 | 932.46 | 20 | 81 | 2.5 |
| YPGPQAEGDSEGLSQGLVDR | 692.53 | 559.29 | 19 | 77 | 5.0 |
| (SEQ ID No. 51) | 692.53 | 774.45 | 19 | 77 | 5.0 |
| | 692.53 | 1188.47 | 17 | 77 | 5.0 |
| ASSIIDELFODR (SEQ ID No. 70) | 697.52 | 922.46 | 22 | 102 | 7.3 |
| | 697.52 | 1035.47 | 21 | 102 | 7.3 |
| | 697.52 | 565.29 | 20 | 102 | 7.3 |
| NTLIIYLDK (SEQ ID No. 14) | 546.92 | 537.96 | 13 | 68 | 6.0 |
| | 546.92 | 877.57 | 15 | 68 | 6.0 |
| VLLDGVQNPR (SEQ ID No. 13) | 555.95 | 670.38 | 21 | 69 | 4.3 |
| | 555.95 | 898.46 | 18 | 69 | 4.3 |
| | 555.95 | 785.23 | 19 | 69 | 4.3 |
| FIPLIPIPER (SEQ ID No. 53) | 597.99 | 467.88 | 14 | 60 | 7.2 |
| | 597.99 | 611.38 | 21 | 60 | 7.2 |
| IVESYQIR (SEQ ID No. 54) | 504.37 | 795.38 | 15 | 57 | 3.9 |
| | 504.37 | 666.38 | 16 | 57 | 3.9 |
| | 504.37 | 894.46 | 18 | 57 | 3.9 |
| LLGHLASHGALR (SEQ ID No. 36) | 415.69 | 509.88 | 12 | 58 | 3.7 |
| | 415.69 | 711.38 | 17 | 58 | 3.7 |
| | 415.69 | 566.43 | 13 | 58 | 3.7 |
| SLQLASWPNR (SEQ ID No. 38) | 586.46 | 659.36 | 17 | 71 | 5.5 |
| | 586.46 | 730.38 | 19 | 71 | 5.5 |
| | 586.46 | 843.46 | 19 | 71 | 5.5 |
| QIDLVDNYFVK (SEQ ID No. 39) | 677.52 | 785.38 | 19 | 107 | 6.4 |
| | 677.52 | 884.46 | 19 | 107 | 6.4 |
| | 677.52 | 1112.54 | 20 | 107 | 6.4 |
| LFCETCDR (SEQ ID No. 40) | 550.84 | 840.29 | 17 | 77 | 3.4 |
| | 550.84 | 420.79 | 15 | 77 | 3.4 |
| | 550.84 | 680.29 | 18 | 77 | 3.4 |
| AGLAASLAGPHSIVGR (SEQ ID | 739.08 | 822.46 | 27 | 114 | 5.0 |
| No. 20) | 739.08 | 893.47 | 27 | 114 | 5.0 |
| | 739.08 | 1093.61 | 28 | 114 | 5.0 |
| AVVVHAGEDDLGR (SEQ ID No. | 446.72 | 584.46 | 11 | 46 | 3.6 |
| 21) | 446.72 | 485.36 | 15 | 46 | 3.6 |
| | 446.72 | 534.85 | 11 | 46 | 3.6 |
| LAVQFTNR (SEQ ID No. 41) | 474.88 | 537.29 | 17 | 59 | 4.2 |
| | 474.88 | 665.38 | 15 | 59 | 4.2 |
| | 474.88 | 764.38 | 15 | 59 | 4.2 |
| ASTLQLGSPR (SEQ ID No. 42) | 515.41 | 416.28 | 17 | 58 | 3.9 |
| | 515.41 | 529.29 | 18 | 58 | 3.9 |
| | 515.41 | 657.38 | 17 | 58 | 3.9 |
| NVVLQTLEGHLR (SEQ ID No. 71) | 690.02 | 825.43 | 25 | 110 | 6.2 |
| | 690.02 | 313.25 | 25 | 110 | 6.2 |
| | 690.02 | 953.53 | 24 | 110 | 6.2 |
| TAEAQLAYELQGAR (SEQ ID No. | 761.03 | 1020.54 | 24 | 116 | 5.5 |
| 73) | 761.03 | 907.46 | 25 | 116 | 5.5 |
| | 761.03 | 836.39 | 24 | 116 | 5.5 |
| ELSEALGQIFDSQR (SEQ ID No. ) | 797.07 | 950.46 | 25 | 129 | 6.8 |
| | 797.07 | 652.36 | 24 | 129 | 6.8 |
| | 797.07 | 390.21 | 36 | 129 | 6.8 |
| | 797.07 | 765.38 | 24 | 129 | 6.8 |
| ASHEEVEGLVEK (SEQ ID No. 15) | 663.95 | 584.88 | 21 | 126 | 3.6 |
| | 663.95 | 296.21 | 26 | 126 | 3.6 |
| | 663.95 | 1031.51 | 23 | 126 | 3.6 |
| EGTYSLPK (SEQ ID No. 118) | 447.86 | 444.29 | 14 | 49 | 3.7 |
| | 447.86 | 357.24 | 13 | 49 | 3.7 |
| SEFVVPDLELPSWLTTGNYR (SEQ | 1162.27 | 364.13 | 44 | 165 | 8.5 |
| ID No. 117) | 1162.27 | 463.28 | 36 | 165 | 8.5 |
| | 1162.27 | 881.54 | 31 | 165 | 8.5 |
| LSELIQPLPLER (SEQ ID No. 90) | 704.59 | 724.46 | 22 | 90 | 6.4 |
| | 704.59 | 330.14 | 27 | 90 | 6.4 |
| YTNWIQK (SEQ ID No. 91) | 476.84 | 688.38 | 14 | 66 | 4.1 |
| | 476.84 | 789.45 | 16 | 66 | 4.1 |
| LASHEYLK (SEQ ID No. 110) | 480.86 | 388.75 | 17 | 97 | 2.8 |
| | 480.86 | 776.38 | 17 | 97 | 2.8 |
| | 480.86 | 345.24 | 20 | 97 | 2.8 |
| SGIIFAGHDK (SEQ ID No. 97) | 522.91 | 674.30 | 18 | 94 | 3.7 |
| | 522.91 | 787.38 | 18 | 94 | 3.7 |
| | 522.91 | 527.28 | 19 | 94 | 3.7 |
| FLNLTENDIQK (SEQ ID No. 106) | 668.04 | 847.47 | 21 | 94 | 5.4 |
| | 668.04 | 375.24 | 22 | 94 | 5.4 |
| | 668.04 | 1074.63 | 21 | 94 | 5.4 |
| LPAPSIDR (SEQ ID No. 107) | 434.86 | 587.38 | 18 | 58 | 3.8 |
| | 434.86 | 378.21 | 15 | 58 | 3.8 |
| | 434.86 | 658.45 | 16 | 58 | 3.8 |
| ENTSDPSLVIAFGR (SEQ ID No. | 753.58 | 959.64 | 25 | 118 | 6.2 |
| 31) | 753.58 | 450.32 | 23 | 118 | 6.2 |
| | 753.58 | 563.41 | 24 | 118 | 6.2 |
| TVESITDIR (SEQ ID No. 32) | 517.44 | 833.54 | 15 | 59 | 4.4 |
| | 517.44 | 704.46 | 16 | 59 | 4.4 |
| | 517.44 | 504.29 | 14 | 59 | 4.4 |
| AFALWSAVTPLTFTR (SEQ ID No. | 841.20 | 734.46 | 20 | 108 | 8.5 |
| 2) | 841.20 | 835.54 | 21 | 108 | 8.5 |
| | 841.20 | 1092.64 | 26 | 108 | 8.5 |
| EVVSPQEFK (SEQ ID No. 76) | 531.73 | 648.38 | 15 | 62 | 4.2 |
| | 531.73 | 735.39 | 14 | 62 | 4.2 |
| | 531.73 | 834.46 | 16 | 62 | 4.2 |
| ALLQVTISLSK (SEQ ID No. 77) | 587.03 | 747.47 | 18 | 70 | 6.4 |
| | 587.03 | 648.46 | 18 | 70 | 6.4 |
| | 587.03 | 494.88 | 15 | 70 | 6.4 |
| LPEPATLGFSAR (SEQ ID No. | 630.04 | 919.61 | 23 | 79 | 5.7 |
| 115) | 630.04 | 340.21 | 21 | 79 | 5.7 |
| | 630.04 | 573.38 | 19 | 79 | 5.7 |
| | 630.04 | 537.36 | 23 | 79 | 5.7 |
| SDLYIGGVAK (SEQ ID No. 55) | 511.94 | 544.38 | 15 | 57 | 4.5 |
| | 511.94 | 707.46 | 14 | 57 | 4.5 |
| | 511.94 | 431.29 | 15 | 57 | 4.5 |
| ITTQITAGAR (SEQ ID No. 56) | 516.44 | 475.29 | 19 | 62 | 3.4 |
| | 516.44 | 588.38 | 19 | 62 | 3.4 |
| | 516.44 | 817.54 | 17 | 62 | 3.4 |
| YEEEVLSR (SEQ ID No. 47) | 512.91 | 732.46 | 16 | 65 | 3.6 |
| | 512.91 | 293.20 | 15 | 65 | 3.6 |
| | 512.91 | 603.38 | 17 | 65 | 3.6 |
| VDAVFQQEHFFHVFSGPR (SEQ | 716.62 | 882.14 | 22 | 114 | 6.5 |
| ID No. 12) | 716.62 | 799.63 | 26 | 114 | 6.5 |
| | 716.62 | 563.36 | 27 | 114 | 6.5 |
| NYTPQLSEAEVER (SEQ ID No. ) | 768.55 | 1157.63 | 24 | 103 | 4.5 |
| | 768.55 | 579.38 | 23 | 103 | 4.5 |
| | 768.55 | 379.21 | 22 | 103 | 4.5 |
| MYATIYELK (SEQ ID No. 92) | 566.45 | 837.54 | 16 | 69 | 5.5 |
| | 566.45 | 766.46 | 16 | 69 | 5.5 |
| | 566.45 | 1000.63 | 16 | 69 | 5.5 |
| VPLQQNFQDNQFQGK (SEQ ID | 896.18 | 204.21 | 36 | 181 | 4.8 |
| No. 93) | 896.18 | 339.24 | 39 | 181 | 4.8 |
| | 896.18 | 479.29 | 35 | 181 | 4.8 |
| AWTVEQLR (SEQ ID No. 86) | 501.91 | 745.46 | 15 | 55 | 5.0 |
| | 501.91 | 258.20 | 14 | 55 | 5.0 |
| | 501.91 | 545.38 | 17 | 55 | 5.0 |
| SEETLDEGPPK (SEQ ID No. 87) | 601.42 | 985.54 | 18 | 77 | 3.2 |
| | 601.42 | 856.53 | 18 | 77 | 3.2 |
| | 601.42 | 642.38 | 18 | 77 | 3.2 |
| ESWEMNSEEK (SEQ ID No. 88) | 634.90 | 737.38 | 19 | 79 | 3.7 |
| | 634.90 | 606.29 | 18 | 79 | 3.7 |
| | 634.90 | 866.45 | 20 | 79 | 3.7 |
| ALELDSNNEK (SEQ ID No. 89) | 566.91 | 706.38 | 16 | 67 | 3.3 |
| | 566.91 | 948.46 | 16 | 67 | 3.3 |
| | 566.91 | 819.46 | 16 | 67 | 3.3 |
| QEILAALEK (SEQ ID No. 23) | 507.94 | 531.38 | 15 | 63 | 5.3 |
| | 507.94 | 460.38 | 14 | 63 | 5.3 |
| EILDAFDK (SEQ ID No. 22) | 475.90 | 595.29 | 13 | 51 | 5.2 |
| | 475.90 | 480.29 | 12 | 51 | 5.2 |
| | 475.90 | 708.45 | 14 | 51 | 5.2 |
| MDSTANEVEAVK (SEQ ID No. | 647.44 | 788.46 | 20 | 86 | 3.5 |
| 94) | 647.44 | 1047.55 | 20 | 86 | 3.5 |
| | 647.44 | 859.53 | 21 | 86 | 3.5 |
| ALAPEYAK (SEQ ID No. 95) | 431.85 | 678.45 | 11 | 46 | 3.2 |
| | 431.85 | 607.38 | 12 | 46 | 3.2 |
| | 431.85 | 381.21 | 20 | 46 | 3.2 |
| SLTMDPHK (SEQ ID No. 58) | 464.85 | 381.22 | 19 | 60 | 2.8 |
| | 464.85 | 728.38 | 15 | 60 | 2.8 |
| | 464.85 | 364.71 | 14 | 60 | 2.8 |
| ALNSIIDVYHK (SEQ ID No. 7) | 425.07 | 544.96 | 10 | 49 | 5.2 |
| | 425.07 | 774.46 | 14 | 49 | 5.2 |
| GNFHAVYR (SEQ ID No. 8) | 482.40 | 508.29 | 20 | 75 | 2.9 |
| | 482.40 | 323.28 | 18 | 75 | 2.9 |
| | 482.40 | 645.38 | 18 | 75 | 2.9 |
| VIEHIMEDLDTNADK (SEQ ID No. | 872.15 | 267.13 | 44 | 115 | 5.1 |
| 9) | 872.15 | 479.29 | 35 | 115 | 5.1 |
| | 872.15 | 1151.54 | 31 | 115 | 5.1 |
| LTWASHEK (SEQ ID No. 10) | 486.39 | 757.38 | 16 | 68 | 3.0 |
| | 486.39 | 571.30 | 18 | 68 | 3.0 |
| | 486.39 | 379.21 | 15 | 68 | 3.0 |
| DSAHGFLK (SEQ ID No. 100) | 437.86 | 380.29 | 14 | 62 | 3.2 |
| | 437.86 | 464.29 | 16 | 62 | 3.2 |
| | 437.86 | 336.72 | 15 | 62 | 3.2 |
| DGAGDVAFVK (SEQ ID No. 101) | 489.90 | 735.46 | 14 | 56 | 4.2 |
| | 489.90 | 464.36 | 14 | 56 | 4.2 |
| | 489.90 | 563.38 | 15 | 56 | 4.2 |
| YLYGSDQLVVR (SEQ ID No. 59) | 657.02 | 1036.63 | 21 | 89 | 5.2 |
| | 657.02 | 518.88 | 17 | 89 | 5.2 |
| | 657.02 | 873.54 | 22 | 89 | 5.2 |
| ILQALDLPAK (SEQ ID No. 60) | 541.47 | 315.28 | 24 | 63 | 5.4 |
| | 541.47 | 428.29 | 14 | 63 | 5.4 |
| | 541.47 | 855.54 | 15 | 63 | 5.4 |
| LTLLAPLNSVFK (SEQ ID No. 113) | 658.59 | 804.54 | 18 | 77 | 7.7 |
| | 658.59 | 875.54 | 19 | 77 | 7.7 |
| | 658.59 | 512.38 | 15 | 77 | 7.7 |
| GDELADSALEIFK (SEQ ID No. | 704.49 | 294.21 | 26 | 159 | 5.4 |
| 114) | 704.49 | 1114.54 | 26 | 159 | 5.4 |
| | 704.49 | 302.13 | 32 | 159 | 5.4 |
| YQSALLPHK (SEQ ID No. 45) | 528.95 | 765.53 | 16 | 70 | 3.7 |
| | 528.95 | 381.29 | 21 | 70 | 3.7 |
| | 528.95 | 292.20 | 16 | 70 | 3.7 |
| VEPLDFGGTQK (SEQ ID No. 24) | 595.94 | 481.79 | 15 | 70 | 4.8 |
| | 595.94 | 962.54 | 16 | 70 | 4.8 |
| | 595.94 | 752.45 | 22 | 70 | 4.8 |
| VLDIIATINK (SEQ ID No. 25) | 550.50 | 887.61 | 15 | 62 | 6.1 |
| | 550.50 | 546.38 | 15 | 62 | 6.1 |
| TVDNFVALATGEK (SEQ ID No. | 683.05 | 788.54 | 20 | 85 | 6.0 |
| 104) | 683.05 | 689.46 | 20 | 85 | 6.0 |
| | 683.05 | 1164.63 | 20 | 85 | 6.0 |
| VYFDLR (SEQ ID No. 105) | 406.84 | 550.36 | 11 | 45 | 5.2 |
| | 406.84 | 713.45 | 13 | 45 | 5.2 |
| | 406.84 | 403.29 | 12 | 45 | 5.2 |
| VNVDEVGGEALGR (SEQ ID No. ) | 657.99 | 758.46 | 22 | 83 | 4.6 |
| | 657.99 | 1002.54 | 22 | 83 | 4.6 |
| VTTVASHTSDSDVPSGVTEVVVK | 772.30 | 507.95 | 18 | 77 | 4.7 |
| (SEQ ID No. 69) | 772.30 | 1014.61 | 18 | 77 | 4.7 |
| | 772.30 | 650.96 | 14 | 77 | 4.7 |
| QDTLPEAGR (SEQ ID No. 37) | 493.87 | 529.29 | 17 | 63 | 2.9 |
| | 493.87 | 743.45 | 18 | 63 | 2.9 |
| SQLVYQSR (SEQ ID No. 16) | 490.88 | 765.46 | 16 | 58 | 3.1 |
| | 490.88 | 553.29 | 16 | 58 | 3.1 |
| | 490.88 | 652.38 | 16 | 58 | 3.1 |
| VTFLGLQHWVPELAR (SEQ ID | 589.52 | 783.63 | 15 | 68 | 7.4 |
| No. 109) | 589.52 | 585.38 | 13 | 68 | 7.4 |
| | 589.52 | 653.54 | 15 | 68 | 7.4 |
| ALQATVGNSYK (SEQ ID No. 33) | 576.45 | 568.00 | 14 | 68 | 3.4 |
| | 576.45 | 839.46 | 18 | 68 | 3.4 |
| | 576.45 | 768.38 | 18 | 68 | 3.4 |
| NPLFDHAALSAPLPAPSSPPALP | 1141.35 | 494.29 | 39 | 159 | 7.4 |
| (SEQ ID No. 116) | 1141.35 | 1083.67 | 29 | 159 | 7.4 |
| | 1141.35 | 1516.78 | 35 | 159 | 7.4 |
| VLEAELLVLR (SEQ ID No. 49) | 577.97 | 942.55 | 19 | 69 | 6.8 |
| | 577.97 | 813.54 | 20 | 69 | 6.8 |
| | 577.97 | 742.46 | 19 | 69 | 6.8 |
| FTVLTESAAK (SEQ ID No. 50) | 533.92 | 818.46 | 16 | 65 | 4.4 |
| | 533.92 | 719.38 | 17 | 65 | 4.4 |
| | 533.92 | 606.29 | 17 | 65 | 4.4 |
| YYAFDLIAQR (SEQ ID No. 11) | 630.46 | 933.54 | 20 | 78 | 6.4 |
| | 630.46 | 862.46 | 20 | 78 | 6.4 |
| | 630.46 | 715.45 | 20 | 78 | 6.4 |
| KPVEHWHQLVEVSR (SEQ ID No. | 582.15 | 808.55 | 19 | 110 | 5.2 |
| 61) | 582.15 | 589.30 | 22 | 110 | 5.2 |
| | 582.15 | 702.45 | 22 | 110 | 5.2 |
| GPTPTPDK (SEQ ID No. 62) | 406.80 | 359.21 | 20 | 47 | 1.8 |
| | 406.80 | 557.29 | 13 | 47 | 1.8 |
| | 406.80 | 256.13 | 13 | 47 | 1.8 |
| HPCTLGPFIHATNALHVR (SEQ ID | 681.23 | 235.13 | 27 | 127 | 5.4 |
| No. ) | 681.23 | 881.54 | 27 | 127 | 5.4 |
| | 681.23 | 810.46 | 26 | 127 | 5.4 |
| ENITIVDISR (SEQ ID No. 46) | 580.45 | 589.36 | 17 | 69 | 5.3 |
| | 580.45 | 803.46 | 18 | 69 | 5.3 |
| LGDTPGVSYSDIAAR (SEQ ID No. | 761.57 | 1135.63 | 28 | 135 | 4.9 |
| 102) | 761.57 | 568.38 | 26 | 135 | 4.9 |
| | 761.57 | 882.46 | 28 | 135 | 4.9 |
| DVHEGQPLLNVK (SEQ ID No. | 450.40 | 342.36 | 11 | 49 | 4.4 |
| 75) | 450.40 | 683.46 | 12 | 49 | 4.4 |
| | 450.40 | 473.38 | 14 | 49 | 4.4 |
| GAGAFGYFEVTHDITK (SEQ ID | 571.82 | 793.05 | 13 | 67 | 6.1 |
| No. 19) | 571.82 | 729.04 | 14 | 67 | 6.1 |
| | 571.82 | 655.47 | 14 | 67 | 6.1 |
| EGHLRSILGTLTVEQIYQDRDQFAK | 973.78 | 631.01 | 42 | 213 | 6.8 |
| (SEQ ID No. 72) | 973.78 | 324.13 | 55 | 213 | 6.8 |
| | 973.78 | 1213.75 | 36 | 213 | 6.8 |
| IYTSPTWSAFVTDSSWSAR (SEQ | 721.55 | 909.39 | 20 | 75 | 7.4 |
| ID No. 17) | 721.55 | 519.29 | 15 | 75 | 7.4 |
| | 721.55 | 808.38 | 19 | 75 | 7.4 |
| GKEDFLSVSDIIDYFR (SEQ ID No. | 635.45 | 600.29 | 17 | 69 | 7.2 |
| 108) | 635.45 | 713.38 | 17 | 69 | 7.2 |
| | 635.45 | 596.38 | 13 | 69 | 7.2 |
| QLSLPETGELDSATLK (SEQ ID No. | 851.60 | 1260.63 | 28 | 130 | 5.9 |
| 3) | 851.60 | 630.95 | 26 | 130 | 5.9 |
| IDSLMDEISFLK (SEQ ID No. 48) | 706.02 | 982.53 | 21 | 91 | 7.7 |
| | 706.02 | 851.46 | 22 | 91 | 7.7 |
| | 706.02 | 316.14 | 25 | 91 | 7.7 |
| ALLLVGDVAQAADVAIEHR (SEQ | 654.55 | 726.47 | 15 | 71 | 7.7 |
| ID No. 103) | 654.55 | 776.04 | 16 | 71 | 7.7 |
| | 654.55 | 832.55 | 16 | 71 | 7.7 |

Table 3 shows experimentally optimised targeted LC-MS/MS conditions to monitor heavy isotope-labelled peptide internal standards listed in Table 1. This is based on ThermoScientific triple quadruple LC-MS/MS platform TSQ Altis. In Table 2, the following abbreviations are used: RF—Radio Frequency.

TABLE 3

| | Precursor | Product | Collision | | |
|---|---|---|---|---|---|
| Internal standard peptide sequence | (m/z) | (m/z) | Energy (V) | RF Lens (V) | RT (mins) |
| DAFARAFQVWSDVTPLR(U-13C6, 15N4)(SEQ | 714.87 | 983.51 | 22 | 82 | 6.5 |
| ID No. 175) | 714.87 | 395.26 | 18 | 82 | 6.5 |
| | 714.87 | 797.43 | 22 | 82 | 6.5 |
| QMYPELQIAR(U-13C6, 15N4)VEQAT (SEQ ID | 629.82 | 836.48 | 21 | 77 | 5.3 |
| No. 162) | 629.82 | 418.74 | 18 | 77 | 5.3 |
| | 629.82 | 999.54 | 21 | 77 | 5.3 |
| DDTPMTLPK(U-13C6, 15N2)GSTEQ (SEQ ID No. | 513.25 | 694.40 | 14 | 54 | 4.3 |
| 161) | 513.25 | 252.17 | 24 | 54 | 4.3 |
| | 513.25 | 332.13 | 12 | 54 | 4.3 |
| IPTTFENGR(U-13C6, 15N4)CIQAN (SEQ ID No. | 522.77 | 466.22 | 17 | 77 | 3.7 |
| 153) | 522.77 | 834.39 | 20 | 77 | 3.7 |
| AATVGSAGQPLQER(U-13C6, 15N4)AQAWG | 754.40 | 652.36 | 22 | 95 | 4.5 |
| (SEQ ID No. 151) | 754.40 | 837.44 | 24 | 95 | 4.5 |
| LGPLVEQGR(U-13C6, 15N4)VRAAT (SEQ ID No. | 489.78 | 598.31 | 20 | 58 | 4.0 |
| 152) | 489.78 | 499.24 | 17 | 58 | 4.0 |
| EDYGKGVIFYLR(U-13C6, 15N4)(SEQ ID No. | 877.51 | 270.21 | 47 | 168 | 6.1 |
| 141) | 877.51 | 311.13 | 47 | 168 | 6.1 |
| | 877.51 | 434.21 | 41 | 168 | 6.1 |
| LVFFAEDVGSNK(U-13C6, 15N2)GAIIG (SEQ ID | 667.34 | 213.15 | 21 | 80 | 5.9 |
| No. 160) | 667.34 | 1121.53 | 19 | 80 | 5.9 |
| | 667.34 | 827.39 | 22 | 80 | 5.9 |
| VGSNKGAIIGLMVGGV(U-13C5, 15N)V(U- | 549.33 | 487.80 | 10 | 59 | 6.8 |
| 13C5, 15N)(SEQ ID No. 158) | 549.33 | 743.40 | 18 | 59 | 6.8 |
| | 549.33 | 242.14 | 19 | 59 | 6.8 |
| VGSNKGAIIGLMVGGV(U-13C5, 15N)V(U- | 641.39 | 525.33 | 10 | 52 | |
| 13C5, 15N)IA (SEQ ID No. 159) | 641.39 | 656.38 | 10 | 52 | |
| | 641.39 | 203.13 | 15 | 52 | |
| DDILPMDLGTFYR(U-13C6, 15N4)EFQNP (SEQ | 783.38 | 1109.53 | 21 | 83 | 7.4 |
| ID No. 137) | 783.38 | 344.14 | 22 | 83 | 7.4 |
| | 783.38 | 457.22 | 17 | 83 | 7.4 |
| | 783.38 | 555.26 | 21 | 83 | 7.4 |
| LTNLKWGLAWVSSQFYNK(U-13C6, 15N4)(SEQ | 797.40 | 244.10 | 35 | 105 | 7.3 |
| ID No. 135) | 797.40 | 881.42 | 25 | 105 | 7.3 |
| | 797.40 | 980.49 | 25 | 105 | 7.3 |
| LSPEDYTLK(U-13C6, 15N2)VSQAG (SEQ ID No. | 537.28 | 437.22 | 14 | 69 | 4.5 |
| 134) | 537.28 | 873.44 | 15 | 69 | 4.5 |
| MYYSAVEPTK(U-13C6, 15N2)DIFTG (SEQ ID | 598.79 | 902.47 | 17 | 79 | 4.0 |
| No. 169) | 598.79 | 353.22 | 25 | 79 | 4.0 |
| | 598.79 | 739.40 | 18 | 79 | 4.0 |
| NPNLKTLLSVGGWNFGSQR(U-13C6, 15N4) | 766.39 | 1018.47 | 24 | 120 | 6.7 |
| (SEQ ID No. 121) | 766.39 | 1204.57 | 25 | 120 | 6.7 |
| | 766.39 | 961.45 | 24 | 120 | 6.7 |
| FSNTDYAVGYMLR(U-13C6, 15N4)LGAPA (SEQ | 773.86 | 649.33 | 23 | 118 | 6.0 |
| ID No. 122) | 773.86 | 819.44 | 26 | 118 | 6.0 |
| | 773.86 | 982.50 | 25 | 118 | 6.0 |
| YPGPQAEGDSEGLSQGLVDR(U- | 695.66 | 569.32 | 19 | 77 | 5.0 |
| 13C6, 15N4)EKGLS (SEQ ID No. 146) | 695.66 | 784.41 | 19 | 77 | 5.0 |
| | 695.66 | 1188.48 | 17 | 77 | 5.0 |
| NTLIIYLDK(U-13C6, 15N2)VSHSE (SEQ ID No. | 550.82 | 546.30 | 13 | 68 | 6.0 |
| 128) | 550.82 | 885.55 | 15 | 68 | 6.0 |
| SVFSKFIPLIPIPER(U-13C6, 15N4)(SEQ ID No. | 602.87 | 472.79 | 14 | 60 | 7.2 |
| 147) | 602.87 | 621.35 | 21 | 60 | 7.2 |
| QIDLVDNYFVK(U-13C6, 15N2)DTSEA (SEQ ID | 681.36 | 793.39 | 19 | 107 | 6.4 |
| No. 139) | 681.36 | 892.46 | 19 | 107 | 6.4 |
| | 681.36 | 1120.57 | 20 | 107 | 6.4 |
| AGLAASLAGPHSIVGR(U-13C6, 15N4)AVVVH | 743.92 | 832.46 | 27 | 114 | 5.0 |
| (SEQ ID No. 131) | 743.92 | 903.50 | 27 | 114 | 5.0 |
| | 743.92 | 1103.61 | 28 | 114 | 5.0 |
| LAVQFTNR(U-13C6, 15N4)NQYCY (SEQ ID No. | 479.77 | 547.28 | 17 | 59 | 4.2 |
| 140) | 479.77 | 675.34 | 15 | 59 | 4.2 |
| | 479.77 | 774.41 | 15 | 59 | 4.2 |
| EGTYSLPK(U-13C6, 15N2)SEFVV (SEQ ID No. | 451.73 | 452.29 | 14 | 49 | 3.7 |
| 178) | 451.73 | 365.26 | 13 | 49 | 3.7 |
| LSELIQPLPLER(U-13C6, 15N4)DCSAN (SEQ ID | 709.41 | 734.44 | 22 | 90 | 6.4 |
| No. 165) | 709.41 | 330.16 | 27 | 90 | 6.4 |
| EVVSPQEFK(U-13C6, 15N2)QGEDA (SEQ ID No. | 535.78 | 656.34 | 15 | 62 | 4.2 |
| 157) | 535.78 | 743.38 | 14 | 62 | 4.2 |
| | 535.78 | 842.40 | 16 | 62 | 4.2 |
| NLDLKSDLYIGGVAK(U-13C6, 15N2)(SEQ ID No. | 515.78 | 552.35 | 15 | 57 | 4.5 |
| 148) | 515.78 | 715.42 | 14 | 57 | 4.5 |
| | 515.78 | 439.27 | 15 | 57 | 4.5 |
| AWTVEQLR(U-13C6, 15N4)SEQLP (SEQ ID No. | 506.77 | 755.42 | 15 | 55 | 5.0 |
| 163) | 506.77 | 258.12 | 14 | 55 | 5.0 |
| | 506.77 | 555.31 | 17 | 55 | 5.0 |
| ESWEMNSEEK(U-13C6, 15N2)LEQST (SEQ ID | 638.77 | 745.33 | 19 | 79 | 3.7 |
| No. 164) | 638.77 | 614.29 | 18 | 79 | 3.7 |
| | 638.77 | 874.37 | 20 | 79 | 3.7 |
| QEILAALEK(U-13C6, 15N2)GCSFL (SEQ ID No. | 511.80 | 539.32 | 15 | 63 | 5.3 |
| 132) | 511.80 | 468.29 | 14 | 63 | 5.3 |
| MDSTANEVEAVK(U-13C6, 15N2)VHSFP (SEQ ID | 651.31 | 796.42 | 20 | 86 | 3.5 |
| No. 167) | 651.31 | 1055.54 | 20 | 86 | 3.5 |
| | 651.31 | 867.46 | 21 | 86 | 3.5 |
| ALNSIIDVYHK(U-13C6, 15N2)YSLIK (SEQ ID No. | 427.57 | 548.79 | 10 | 49 | 5.2 |
| 123) | 427.57 | 782.42 | 14 | 49 | 5.2 |
| YSLIKGNFHAVYR(U-13C6, 15N4)(SEQ ID No. | 487.24 | 518.29 | 20 | 75 | 2.9 |
| 124) | 487.24 | 328.18 | 18 | 75 | 2.9 |
| | 487.24 | 655.35 | 18 | 75 | 2.9 |
| VIEHIMEDLDTNADK(U-13C6, 15N2)QLSFE (SEQ | 875.92 | 267.12 | 44 | 115 | 5.1 |
| ID No. 125) | 875.92 | 479.29 | 35 | 115 | 5.1 |
| | 875.92 | 1151.48 | 31 | 115 | 5.1 |
| LTWASHEK(U-13C6, 15N2)MHEGD (SEQ ID No. | 490.25 | 765.37 | 16 | 68 | 3.0 |
| 126) | 490.25 | 579.29 | 18 | 68 | 3.0 |
| | 490.25 | 383.19 | 15 | 68 | 3.0 |
| DLLFKDSAHGFLK(U-13C6, 15N2)(SEQ ID No. | 441.73 | 384.21 | 14 | 62 | 3.2 |
| 170) | 441.73 | 472.30 | 16 | 62 | 3.2 |
| | 441.73 | 340.70 | 15 | 62 | 3.2 |
| SGSERLTLLAPLNSVFK(U-13C6, 15N2)(SEQ ID | 662.40 | 812.47 | 18 | 77 | 7.7 |
| No. 176) | 662.40 | 883.51 | 19 | 77 | 7.7 |
| | 662.40 | 512.37 | 15 | 77 | 7.7 |
| YYKVEPLDFGGTQK(U-13C6, 15N2)(SEQ ID No. | 599.81 | 485.75 | 15 | 70 | 4.8 |
| 133) | 599.81 | 970.50 | 16 | 70 | 4.8 |
| | 599.81 | 760.37 | 22 | 70 | 4.8 |
| TVDNFVALATGEK(U-13C6, 15N2)GFGYK (SEQ | 686.86 | 796.46 | 20 | 85 | 6.0 |
| ID No. 172) | 686.86 | 697.39 | 20 | 85 | 6.0 |
| | 686.86 | 1172.60 | 20 | 85 | 6.0 |
| ALWGKVNVDEVGGEALGR(U-13C6, 15N4)(SEQ | 662.84 | 768.42 | 22 | 83 | 4.6 |
| ID No. 119) | 662.84 | 1012.49 | 22 | 83 | 4.6 |
| VTTVASHTSDSDVPSGVTEVVVK(U- | 774.73 | 1022.59 | 18 | 77 | 4.7 |
| 13C6, 15N2)LFDSD (SEQ ID No. 154) | 774.73 | 511.80 | 18 | 77 | 4.7 |
| | 774.73 | 650.80 | 14 | 77 | 4.7 |
| QDTLPEAGR(U-13C6, 15N4)RSEAE (SEQ ID No. | 498.75 | 539.28 | 17 | 63 | 2.9 |
| 138) | 498.75 | 753.41 | 18 | 63 | 2.9 |
| SQLVYQSR(U-13C6, 15N4)RGPLV (SEQ ID No. | 495.76 | 775.43 | 16 | 58 | 3.1 |
| 129) | 495.76 | 563.28 | 16 | 58 | 3.1 |
| | 495.76 | 662.34 | 16 | 58 | 3.1 |
| ASSVRVTFLGLQHWVPELAR(U-13C6, 15N4) | 592.66 | 788.43 | 15 | 68 | 7.4 |
| (SEQ ID No. 174) | 592.66 | 595.34 | 13 | 68 | 7.4 |
| | 592.66 | 658.36 | 15 | 68 | 7.4 |
| ALQATVGNSYK(U-13C6, 15N2)CNAEE (SEQ ID | 580.31 | 576.28 | 14 | 68 | 3.4 |
| No. 136) | 580.31 | 847.44 | 18 | 68 | 3.4 |
| | 580.31 | 776.40 | 18 | 68 | 3.4 |
| EMEVRNPLFDHAALSAPLPAPSSPPAL(U- | 1147.75 | 507.32 | 39 | 159 | 7.4 |
| 13C6, 15N)P(U-13C5, 15N)(SEQ ID No. 177) | 1147.75 | 1090.09 | 29 | 159 | 7.4 |
| | 1147.75 | 1516.78 | 35 | 159 | 7.4 |
| EQQNKVLEAELLVLR(U-13C6, 15N4)(SEQ ID | 582.86 | 952.57 | 19 | 69 | 6.8 |
| No. 144) | 582.86 | 823.52 | 20 | 69 | 6.8 |
| | 582.86 | 752.49 | 19 | 69 | 6.8 |
| FTVLTESAAK(U-13C6, 15N2)NTDAV (SEQ ID | 537.79 | 826.47 | 16 | 65 | 4.4 |
| No. 145) | 537.79 | 727.40 | 17 | 65 | 4.4 |
| | 537.79 | 614.32 | 17 | 65 | 4.4 |
| FSGPRYYAFDLIAQR(U-13C6, 15N4)(SEQ ID No. | 635.32 | 943.52 | 20 | 78 | 6.4 |
| 127) | 635.32 | 872.48 | 20 | 78 | 6.4 |
| | 635.32 | 725.41 | 20 | 78 | 6.4 |
| LANPRKPVEHWHQLVEVSR(U-13C6, 15N2) | 587.98 | 813.42 | 19 | 110 | 5.2 |
| (SEQ ID No. 150) | 587.98 | 599.33 | 22 | 110 | 5.2 |
| | 587.98 | 712.42 | 22 | 110 | 5.2 |
| ENITIVDISR(U-13C6, 15N4)KINQL (SEQ ID No. | 585.32 | 599.33 | 17 | 69 | 5.3 |
| 142) | 585.32 | 813.47 | 18 | 69 | 5.3 |
| DVHEGQPLLNVK(U-13C6, 15N4)FSNCN (SEQ ID | 452.91 | 346.23 | 11 | 49 | 4.4 |
| No. 156) | 452.91 | 691.45 | 12 | 49 | 4.4 |
| VVHAKGAGAFGYFEVTHDITK(U-13C6, 15N2) | 574.38 | 796.99 | 13 | 67 | 6.1 |
| (SEQ ID No. 130) | 574.38 | 732.96 | 14 | 67 | 6.1 |
| EGHLRSILGTLTVEQIYQDR(U- | 977.06 | 631.01 | 42 | 213 | 6.8 |
| 13C6, 15N4)DQFAK (SEQ ID No. 155) | 977.06 | 324.13 | 55 | 213 | 6.8 |
| | 977.06 | 1218.80 | 36 | 213 | 6.8 |
| GKEDFLSVSDIIDYFR(U-13C6, 15N4)KMPLL | 638.65 | 610.28 | 17 | 69 | 7.2 |
| (SEQ ID No. 173) | 638.65 | 723.37 | 17 | 69 | 7.2 |
| LLLQKQLSLPETGELDSATLK(U-13C6, 15N2)(SEQ | 855.45 | 1268.64 | 28 | 130 | 5.9 |
| ID No. 120) | 855.45 | 634.82 | 26 | 130 | 5.9 |
| IDSLMDEISFLK(U-13C6, 15N2)KVHEE (SEQ ID | 709.87 | 990.50 | 21 | 91 | 7.7 |
| No. 143) | 709.87 | 859.46 | 22 | 91 | 7.7 |
| ALLLVGDVAQAADVAIEHR(U- | 657.80 | 731.36 | 15 | 71 | 7.7 |
| 13C6, 15N4)NEAEL (SEQ ID No. 171) | 657.80 | 780.90 | 16 | 71 | 7.7 |

1. A method for assessing the efficacy of a treatment for a neurodegenerative disease in a subject, the method comprising:
(i) preparing a biological sample from the subject for assay by incubating the sample with a protease to form a proteolytic digest of proteins in the sample; and
(ii) assaying the proteolytic digest of proteins of step (i) for the presence of a proteolytic peptide of at least one protein selected from the group of proteins consisting of Matrix metalloproteinase-9, Chitinase-3-like protein 1, Protein S100-A8, Protein S100-A9, Neutrophil collagenase (MMP8). Complement C3, Galectin-3-binding protein, Catalase, Extracellular superoxide dismutase, Prosaposin, Beta-hexosaminidase subunit beta, Cathepsin D, Beclin-1, Lysosome-associated membrane glycoprotein 1, Autophagy protein 13, E3 ubiquitin-protein ligase RNF26, E3 ubiquitin-protein ligase TRIM33, FAST kinase domain-containing protein 5, mitochondrial, Apoptosis-inducing factor 1 mitochondrial, Transmembrane protein 126A, Neurofilament light polypeptide, Chromogranin-A, Contactin-1, Neurexin-1-beta, Protein MTSS 1 (Metastasis suppressor YGL-1), Synaptotagmin-3, Apolipoprotein E, Apolipoprotein D, Clusterin, Flotillin-2, Cadherin-2, Neural cell adhesion molecule 2, Amyloid-beta precursor protein, Amyloid-like protein 1, Peptidyl-prolyl cis-trans isomerase FKBP3, Peptidyl-prolyl cis-trans isomerase FKBP4, Kallikrein-6, Neutrophil gelatinase-associated lipocalin, Protein disulfide-isomerase A1 (PDIA1), Limbic system-associated membrane protein (Fragment), Ceruloplasmin, Serotransferrin, Vacuolar protein sorting-associated protein 16 homolog (hVPS16), Peptidyl-prolyl cis-trans isomerase (PPlase; HEL-S-39), Lymphocyte cytosolic protein 2, Lactadherin, 72 kDa type IV collagenase, Transforming growth factor-beta-induced protein ig-h3, Neural proliferation differentiation and control protein 1, Ganglioside GM2 activator,
wherein the presence of said proteolytic peptide is assayed for using mass spectrometry with reference to a corresponding labelled and/or unlabelled reference proteolytic peptide.

2. The method of claim 1 further comprising:
stratifying the subject according to the relative concentration of a peptide measured in the sample.

3. The method of claim 1 further comprising:
diagnosing the neurodegenerative disease of the subject according to the relative concentration of a peptide measured in the sample.

4. The method of claim 1 further comprising:
administering a pharmaceutical composition to the subject for the treatment of the neurodegenerative disease.

5. The method of claim 1 further comprising:
(b) treating the subject with a pharmaceutical composition for a second-line treatment of the neurodegenerative disease.

6. The method of claim 1 wherein the protease is a serine protease, a cysteine protease, a threonine protease, an aspartic protease, a glutamic protease or a metalloprotease.

7. The method of claim 1 wherein the protease is trypsin.

8. A kit for assessing the efficacy of a first-line treatment for a neurodegenerative disease in a subject, comprising:
a plurality of sample preparation media for analysis of a sample by mass spectrometry for the presence of a proteolytic peptide of at least one protein selected from the group of proteins consisting of Matrix metalloproteinase-9, Chitinase-3-like protein 1, Protein S100-A8, Protein S100-A9, Neutrophil collagenase (MMP8). Complement C3, Galectin-3-binding protein, Catalase, Extracellular superoxide dismutase, Prosaposin, Beta-hexosaminidase subunit beta, Cathepsin D, Beclin-1, Lysosome-associated membrane glycoprotein 1, Autophagy protein 13, E3 ubiquitin-protein ligase RNF26, E3 ubiquitin-protein ligase TRIM33, FAST kinase domain-containing protein 5, mitochondrial, Apoptosis-inducing factor 1 mitochondrial, Transmembrane protein 126A, Neurofilament light polypeptide, Chromogranin-A, Contactin-1, Neurexin-1-beta, Protein MTSS 1 (Metastasis suppressor YGL-1), Synaptotagmin-3, Apolipoprotein E, Apolipoprotein D, Clusterin, Flotillin-2, Cadherin-2, Neural cell adhesion molecule 2, Amyloid-beta precursor protein, Amyloid-like protein 1, Peptidyl-prolyl cis-trans isomerase FKBP3, Peptidyl-prolyl cis-trans isomerase FKBP4, Kallikrein-6, Neutrophil gelatinase-associated lipocalin, Protein disulfide-isomerase A1 (PDIA1), Limbic system-associated membrane protein (Fragment), Ceruloplasmin, Serotransferrin, Vacuolar protein sorting-associated protein 16 homolog (hVPS16), Peptidyl-prolyl cis-trans isomerase (PPlase; HEL-S-39), Lymphocyte cytosolic protein 2, Lactadherin, 72 kDa type IV collagenase, Transforming growth factor-beta-induced protein ig-h3, Neural proliferation differentiation and control protein 1, Ganglioside GM2 activator.

9. The kit according to claim 8, wherein the sample preparation media comprises a protease, optionally wherein the protease is a serine protease, a cysteine protease, a threonine protease, an aspartic protease, a glutamic protease or a metalloprotease.

10. The kit according to claim 9 wherein the protease is trypsin.

11. The method of claim 1 wherein the neurodegenerative disease is selected from the group consisting of Parkinson's Disease, Alzheimer's disease, Huntington's disease, Multiple Sclerosis (MS), Amyotrophic lateral sclerosis (ALS), Batten disease or a transmissible spongiform encephalopathy (e.g. scrapie, bovine spongiform encephalopathy (BSE) or Creutzfeldt-Jakob disease (CJD), (including iatrogenic, variant, familial or sporadic forms of CJD), or a lysosomal neurodegenerative disorder (e.g. Gaucher's disease).

12. The method of claim 1 wherein the neurodegenerative disease is Parkinson's Disease.

13. The method of claim 1 wherein the sample is selected from the group consisting of cerebrospinal fluid (CSF), blood (serum, whole blood (venous blood or peripheral blood), plasma, urine, tear, saliva, interstitial fluid, lymph fluid or tissue samples.

14. The method of claim 1 wherein the sample is cerebrospinal fluid (CSF).

15. The method of claim 1 wherein the proteolytic peptide of at least one protein is selected from the group consisting of:
(SEQ ID No. 1)
VNVDEVGGEALGR
(SEQ ID No. 2)
AFALWSAVTPLTFTR
(SEQ ID No. 3)
QLSLPETGELDSATLK
(SEQ ID No. 4)
LVMGIPTFGR
(SEQ ID No. 5)
TLLSVGGWNFGSQR
(SEQ ID No. 6)
FSNTDYAVGYMLR
(SEQ ID No. 7)
ALNSIIDVYHK
(SEQ ID No. 8)
GNFHAVYR
(SEQ ID No. 9)
VIEHIMEDLDTNADK
(SEQ ID No. 10)
LTWASHEK
(SEQ ID No. 11)
YYAFDLIAQR
(SEQ ID No. 12)
VDAVFQQEHFFHVFSGPR
(SEQ ID No. 13)
VLLDGVQNPR
(SEQ ID No. 14)
NTLIIYLDK
(SEQ ID No. 15)
ASHEEVEGLVEK
(SEQ ID No. 16)
SQLVYQSR
(SEQ ID No. 17)
IYTSPTWSAFVTDSSWSAR
(SEQ ID No. 18)
FSTVAGESGSADTVR
(SEQ ID No. 19)
GAGAFGYFEVTHDITK
(SEQ ID No. 20)
AGLAASLAGPHSIVGR
(SEQ ID No. 21)
AVVVHAGEDDLGR
(SEQ ID No. 22)
EILDAFDK
(SEQ ID No. 23)
QEILAALEK
(SEQ ID No. 24)
VEPLDFGGTQK
(SEQ ID No. 25)
VLDIIATINK
(SEQ ID No. 26)
LSPEDYTLK
(SEQ ID No. 27)
VSTLPAITLK
(SEQ ID No. 28)
WGLAWVSSQFYNK
(SEQ ID No. 29)
TSNNSTMQVSFVCQR
(SEQ ID No. 30)
ELALEEER
(SEQ ID No. 31)
ENTSDPSLVIAFGR
(SEQ ID No. 32)
TVESITDIR
(SEQ ID No. 33)
ALQATVGNSYK
(SEQ ID No. 34)
DDILPMDLGTFYR
(SEQ ID No. 35)
TVQVIVQAR
(SEQ ID No. 36)
LLGHLASHGALR
(SEQ ID No. 37)
QDTLPEAGR
(SEQ ID No. 38)
SLQLASWPNR
(SEQ ID No. 39)
QIDLVDNYFVK
(SEQ ID No. 40)
LFCETCDR
(SEQ ID No. 41)
LAVQFTNR
(SEQ ID No. 42)
ASTLQLGSPR
(SEQ ID No. 43)
GVIFYLR
(SEQ ID No. 44)
ELWFSDDPNVTK
(SEQ ID No. 45)
YQSALLPHK
(SEQ ID No. 46)
ENITIVDISR
(SEQ ID No. 47)
YEEEVLSR
(SEQ ID No. 48)
IDSLMDEISFLK
(SEQ ID No. 49)
VLEAELLVLR
(SEQ ID No. 50)
FTVLTESAAK
(SEQ ID No. 51)
YPGPQAEGDSEGLSQGLVDR
(SEQ ID No. 52)
SGELEQEEER
(SEQ ID No. 53)
FIPLIPIPER
(SEQ ID No. 54)
IVESYQIR
(SEQ ID No. 55)
SDLYIGGVAK
(SEQ ID No. 56)
ITTQITAGAR
(SEQ ID No. 57)
TTVVAAAAFLDAFQK
(SEQ ID No. 58)
SLTMDPHK
(SEQ ID No. 59)
YLYGSDQLVVR
(SEQ ID No. 60)
ILQALDLPAK
(SEQ ID No. 61)
KPVEHWHQLVEVSR
(SEQ ID No. 62)
GPTPTPDK
(SEQ ID No. 63)
HPC(Carbamidomethyl)TLGPFIHATNALHVR
(SEQ ID No. 64)
AATVGSLAGQPLQER
(SEQ ID No. 65)
GEVQAMLGQSTEELR
(SEQ ID No. 66)
LGPLVEQGR
(SEQ ID No. 67)
IPTTFENGR
(SEQ ID No. 68)
VLNQELR
(SEQ ID No. 69)
VTTVASHTSDSDVPSGVTEVVVK
(SEQ ID No. 70)
ASSIIDELFQDR
(SEQ ID No. 71)
NVVLQTLEGHLR
(SEQ ID No. 72)
EGHLRSILGTLTVEQIYQDRDQFAK
(SEQ ID No. 73)
TAEAQLAYELQGAR
(SEQ ID No. 74)
GPFPQELVR
(SEQ ID No. 75)
DVHEGQPLLNVK
(SEQ ID No. 76)
EVVSPQEFK
(SEQ ID No. 77)
ALLQVTISLSK
(SEQ ID No. 78)
QQLVETHMAR
(SEQ ID No. 79)
GAIIGLMVGGVV
(SEQ ID No. 80)
GAIIGLMVGGVVIA
(SEQ ID No. 81)
LVFFAEDVGSNK
(SEQ ID No. 82)
WYFDVTEGK
(SEQ ID No. 83)
DDTPMTLPK
(SEQ ID No. 84)
HGYENPTYR
(SEQ ID No. 85)
QMYPELQIAR
(SEQ ID No. 86)
AWTVEQLR
(SEQ ID No. 87)
SEETLDEGPPK
(SEQ ID No. 88)
ESWEMNSEEK
(SEQ ID No. 89)
ALELDSNNEK
(SEQ ID No. 90)
LSELIQPLPLER
(SEQ ID No. 91)
YTNWIQK
(SEQ ID No. 92)
MYATIYELK
(SEQ ID No. 93)
VPLQQNFQDNQFQGK
(SEQ ID No. 94)
MDSTANEVEAVK
(SEQ ID No. 95)
ALAPEYAK
(SEQ ID No. 96)
VTVNYPPTITESK
(SEQ ID No. 97)
SGIIFAGHDK
(SEQ ID No. 98)
MYYSAVEPTK
(SEQ ID No. 99)
IYHSHIDAPK
(SEQ ID No. 100)
DSAHGFLK
(SEQ ID No. 101)
DGAGDVAFVK
(SEQ ID No. 102)
LGDTPGVSYSDIAAR
(SEQ ID No. 103)
ALLLVGDVAQAADVAIEHR
(SEQ ID No. 104)
TVDNFVALATGEK
(SEQ ID No. 105)
VYFDLR
(SEQ ID No. 106)
FLNLTENDIQK
(SEQ ID No. 107)
LPAPSIDR
(SEQ ID No. 108)
GKEDFLSVSDIIDYFR
(SEQ ID No. 109)
VTFLGLQHWVPELAR
(SEQ ID No. 110)
LASHEYLK
(SEQ ID No. 111)
AFQVWSDVTPLR
(SEQ ID No. 112)
FFGLPQTGDLDQNTIETMR
(SEQ ID No. 113)
LTLLAPLNSVFK
(SEQ ID No. 114)
GDELADSALEIFK
(SEQ ID No. 115)
LPEPATLGFSAR
(SEQ ID No. 116)
NPLFDHAALSAPLPAPSSPPALP
(SEQ ID No. 117)
SEFVVPDLELPSWLTTGNYR
and
(SEQ ID No. 118)
EGTYSLPK.

16. The method of claim 1 wherein the at least one protein is selected from the group consisting of Chromogranin-A, Amyloid-beta Precursor Protein, Amyloid-like Protein 1, Transforming Growth Factor-Beta-Induced Protein IG-H3, Prosaposin and Apolipoprotein E.

17. The method of claim 1 wherein the proteolytic peptide of at least one protein is selected from the group consisting of:
(SEQ ID No. 52)
SGELEQEEER
(SEQ ID No. 78)
QQLVETHMAR
(SEQ ID No. 85)
QMYPELQIAR
(SEQ ID No. 114)
GDELADSALEIFK
(SEQ ID No. 23)
QEILAALEK
and
(SEQ ID No. 64)
AATVGSLAGQPLQER.

18. The method of claim 1 wherein the at least one protein is selected from the group consisting of:
Matrix metalloproteinase-9
Chitinase-3-like protein 1
Protein S100-A8
Protein S100-A9
Neutrophil collagenase (MMP8)
Complement C3
Galectin-3-binding protein
Catalase
Extracellular superoxide dismutase
Prosaposin
Beta-hexosaminidase subunit beta
Cathepsin D
Beclin-1
Lysosome-associated membrane glycoprotein 1
Autophagy protein 13
E3 ubiquitin-protein ligase RNF26
E3 ubiquitin-protein ligase TRIM33
FAST kinase domain-containing protein 5, mitochondrial
Apoptosis-inducing factor 1, mitochondrial
Transmembrane protein 126A
Neurofilament light polypeptide
Chromogranin-A
Contactin-1
Neurexin-1-beta
Synaptotagmin-3
Apolipoprotein E
Apolipoprotein D
Clusterin
Flotillin-2
Cadherin-2
Neural cell adhesion molecule 2
Amyloid-beta precursor protein
Amyloid-like protein 1
Peptidyl-prolyl cis-trans isomerase FKBP3
Peptidyl-prolyl cis-trans isomerase FKBP4
Kallikrein-6
Neutrophil gelatinase-associated lipocalin
Protein disulfide-isomerase A1 (PDIA1)
Limbic system-associated membrane protein (Fragment)
Ceruloplasmin
Serotransferrin
Vacuolar protein sorting-associated protein 16 homolog (hVPS16)
Peptidyl-prolyl cis-trans isomerase (PPlase; HEL-S-39)
Lactadherin
72 kDa type IV collagenase
Transforming growth factor-beta-induced protein ig-h3
Neural proliferation differentiation and control protein 1 and
Ganglioside GM2 activator.

19. The method of claim 18 wherein the at least one protein is all 48 of the proteins.

20. The method of claim 1 wherein the at least one protein is selected from the consisting of E3 Ubiquitin-Protein Ligase TRIM33, Fast Kinase Domain-Containing Protein 5, Mitochondrial and Contactin-1.

21. The method of claim 20 wherein the proteolytic peptide of E3 Ubiquitin-Protein Ligase TRIM33 is LFCETCDR (SEQ ID No. 40); the proteolytic peptide of Fast Kinase Domain-Containing Protein 5, Mitochondrial is LAVQFTNR (SEQ ID No. 41); and/or the proteolytic peptide of Contactin-1 is IVESYQIR (SEQ ID No. 54).


SEQUENCE LISTING (178 sequences)

SEQ ID NO: 1 - protein, 13 aa, Homo sapiens
VNVDEVGGEALGR

SEQ ID NO: 2 - protein, 15 aa, Homo sapiens
AFALWSAVTPLTFTR

SEQ ID NO: 3 - protein, 16 aa, Homo sapiens
QLSLPETGELDSATLK

SEQ ID NO: 4 - protein, 10 aa, Homo sapiens
LVMGIPTFGR

SEQ ID NO: 5 - protein, 14 aa, Homo sapiens
TLLSVGGWNFGSQR

SEQ ID NO: 6 - protein, 13 aa, Homo sapiens
FSNTDYAVGYMLR

SEQ ID NO: 7 - protein, 11 aa, Homo sapiens
ALNSIIDVYHK

SEQ ID NO: 8 - protein, 8 aa, Homo sapiens
GNFHAVYR

SEQ ID NO: 9 - protein, 15 aa, Homo sapiens
VIEHIMEDLDTNADK

SEQ ID NO: 10 - protein, 8 aa, Homo sapiens
LTWASHEK

SEQ ID NO: 11 - protein, 10 aa, Homo sapiens
YYAFDLIAQR

SEQ ID NO: 12 - protein, 18 aa, Homo sapiens
VDAVFQQEHFFHVFSGPR

SEQ ID NO: 13 - protein, 10 aa, Homo sapiens
VLLDGVQNPR

SEQ ID NO: 14 - protein, 9 aa, Homo sapiens
NTLIIYLDK

SEQ ID NO: 15 - protein, 12 aa, Homo sapiens
ASHEEVEGLVEK

SEQ ID NO: 16 - protein, 8 aa, Homo sapiens
SQLVYQSR

SEQ ID NO: 17 - protein, 19 aa, Homo sapiens
IYTSPTWSAFVTDSSWSAR

SEQ ID NO: 18 - protein, 15 aa, Homo sapiens
FSTVAGESGSADTVR

SEQ ID NO: 19 - protein, 16 aa, Homo sapiens
GAGAFGYFEVTHDITK

SEQ ID NO: 20 - protein, 16 aa, Homo sapiens
AGLAASLAGPHSIVGR

SEQ ID NO: 21 - protein, 13 aa, Homo sapiens
AVVVHAGEDDLGR

SEQ ID NO: 22 - protein, 8 aa, Homo sapiens
EILDAFDK

SEQ ID NO: 23 - protein, 9 aa, Homo sapiens
QEILAALEK

SEQ ID NO: 24 - protein, 11 aa, Homo sapiens
VEPLDFGGTQK

SEQ ID NO: 25 - protein, 10 aa, Homo sapiens
VLDIIATINK

SEQ ID NO: 26 - protein, 9 aa, Homo sapiens
LSPEDYTLK

SEQ ID NO: 27 - protein, 10 aa, Homo sapiens
VSTLPAITLK

SEQ ID NO: 28 - protein, 13 aa, Homo sapiens
WGLAWVSSQFYNK

SEQ ID NO: 29 - protein, 15 aa, Homo sapiens
TSNNSTMQVSFVCQR

SEQ ID NO: 30 - protein, 8 aa, Homo sapiens
ELALEEER

SEQ ID NO: 31 - protein, 14 aa, Homo sapiens
ENTSDPSLVIAFGR

SEQ ID NO: 32 - protein, 9 aa, Homo sapiens
TVESITDIR

SEQ ID NO: 33 - protein, 11 aa, Homo sapiens
ALQATVGNSYK

SEQ ID NO: 34 - protein, 13 aa, Homo sapiens
DDILPMDLGTFYR

SEQ ID NO: 35 - protein, 9 aa, Homo sapiens
TVQVIVQAR

SEQ ID NO: 36 - protein, 12 aa, Homo sapiens
LLGHLASHGALR

SEQ ID NO: 37 - protein, 9 aa, Homo sapiens
QDTLPEAGR

SEQ ID NO: 38 - protein, 10 aa, Homo sapiens
SLQLASWPNR

SEQ ID NO: 39 - protein, 11 aa, Homo sapiens
QIDLVDNYFVK

SEQ ID NO: 40 - protein, 8 aa, Homo sapiens
LFCETCDR

SEQ ID NO: 41 - protein, 8 aa, Homo sapiens
LAVQFTNR

SEQ ID NO: 42 - protein, 10 aa, Homo sapiens
ASTLQLGSPR

SEQ ID NO: 43 - protein, 7 aa, Homo sapiens
GVIFYLR

SEQ ID NO: 44 - protein, 12 aa, Homo sapiens
ELWFSDDPNVTK

SEQ ID NO: 45 - protein, 9 aa, Homo sapiens
YQSALLPHK

SEQ ID NO: 46 - protein, 10 aa, Homo sapiens
ENITIVDISR

SEQ ID NO: 47 - protein, 8 aa, Homo sapiens
YEEEVLSR

SEQ ID NO: 48 - protein, 12 aa, Homo sapiens
IDSLMDEISFLK

SEQ ID NO: 49 - protein, 10 aa, Homo sapiens
VLEAELLVLR

SEQ ID NO: 50 - protein, 10 aa, Homo sapiens
FTVLTESAAK

SEQ ID NO: 51 - protein, 20 aa, Homo sapiens
YPGPQAEGDSEGLSQGLVDR

SEQ ID NO: 52 - protein, 10 aa, Homo sapiens
SGELEQEEER

SEQ ID NO: 53 - protein, 10 aa, Homo sapiens
FIPLIPIPER

SEQ ID NO: 54 - protein, 8 aa, Homo sapiens
IVESYQIR

SEQ ID NO: 55 - protein, 10 aa, Homo sapiens
SDLYIGGVAK

SEQ ID NO: 56 - protein, 10 aa, Homo sapiens
ITTQITAGAR

SEQ ID NO: 57 - protein, 15 aa, Homo sapiens
TTVVAAAAFLDAFQK

SEQ ID NO: 58 - protein, 8 aa, Homo sapiens
SLTMDPHK

SEQ ID NO: 59 - protein, 11 aa, Homo sapiens
YLYGSDQLVVR

SEQ ID NO: 60 - protein, 10 aa, Homo sapiens
ILQALDLPAK

SEQ ID NO: 61 - protein, 14 aa, Homo sapiens
KPVEHWHQLVEVSR

SEQ ID NO: 62 - protein, 8 aa, Homo sapiens
GPTPTPDK

SEQ ID NO: 63 - protein, 18 aa, Homo sapiens
  MISC_FEATURE (2)..(4): Carbamidomethyl
HPCTLGPFIHATNALHVR

SEQ ID NO: 64 - protein, 15 aa, Homo sapiens
AATVGSLAGQPLQER

SEQ ID NO: 65 - protein, 15 aa, Homo sapiens
GEVQAMLGQSTEELR

SEQ ID NO: 66 - protein, 9 aa, Homo sapiens
LGPLVEQGR

SEQ ID NO: 67 - protein, 9 aa, Homo sapiens
IPTTFENGR

SEQ ID NO: 68 - protein, 7 aa, Homo sapiens
VLNQELR

SEQ ID NO: 69 - protein, 23 aa, Homo sapiens
VTTVASHTSDSDVPSGVTEVVVK

SEQ ID NO: 70 - protein, 12 aa, Homo sapiens
ASSIIDELFQDR

SEQ ID NO: 71 - protein, 12 aa, Homo sapiens
NVVLQTLEGHLR

SEQ ID NO: 72 - protein, 25 aa, Homo sapiens
EGHLRSILGTLTVEQIYQDRDQFAK

SEQ ID NO: 73 - protein, 14 aa, Homo sapiens
TAEAQLAYELQGAR

SEQ ID NO: 74 - protein, 9 aa, Homo sapiens
GPFPQELVR

SEQ ID NO: 75 - protein, 12 aa, Homo sapiens
DVHEGQPLLNVK

SEQ ID NO: 76 - protein, 9 aa, Homo sapiens
EVVSPQEFK

SEQ ID NO: 77 - protein, 11 aa, Homo sapiens
ALLQVTISLSK

SEQ ID NO: 78 - protein, 10 aa, Homo sapiens
QQLVETHMAR

SEQ ID NO: 79 - protein, 12 aa, Homo sapiens
GAIIGLMVGGVV

SEQ ID NO: 80 - protein, 14 aa, Homo sapiens
GAIIGLMVGGVVIA

SEQ ID NO: 81 - protein, 12 aa, Homo sapiens
LVFFAEDVGSNK

SEQ ID NO: 82 - protein, 9 aa, Homo sapiens
WYFDVTEGK

SEQ ID NO: 83 - protein, 9 aa, Homo sapiens
DDTPMTLPK

SEQ ID NO: 84 - protein, 9 aa, Homo sapiens
HGYENPTYR

SEQ ID NO: 85 - protein, 10 aa, Homo sapiens
QMYPELQIAR

SEQ ID NO: 86 - protein, 8 aa, Homo sapiens
AWTVEQLR

SEQ ID NO: 87 - protein, 11 aa, Homo sapiens
SEETLDEGPPK

SEQ ID NO: 88 - protein, 10 aa, Homo sapiens
ESWEMNSEEK

SEQ ID NO: 89 - protein, 10 aa, Homo sapiens
ALELDSNNEK

SEQ ID NO: 90 - protein, 12 aa, Homo sapiens
LSELIQPLPLER

SEQ ID NO: 91 - protein, 7 aa, Homo sapiens
YTNWIQK

SEQ ID NO: 92 - protein, 9 aa, Homo sapiens
MYATIYELK

SEQ ID NO: 93 - protein, 15 aa, Homo sapiens
VPLQQNFQDNQFQGK

SEQ ID NO: 94 - protein, 12 aa, Homo sapiens
MDSTANEVEAVK

SEQ ID NO: 95 - protein, 8 aa, Homo sapiens
ALAPEYAK

SEQ ID NO: 96 - protein, 13 aa, Homo sapiens
VTVNYPPTITESK

SEQ ID NO: 97 - protein, 10 aa, Homo sapiens
SGIIFAGHDK

SEQ ID NO: 98 - protein, 10 aa, Homo sapiens
MYYSAVEPTK

SEQ ID NO: 99 - protein, 10 aa, Homo sapiens
IYHSHIDAPK

SEQ ID NO: 100 - protein, 8 aa, Homo sapiens
DSAHGFLK

SEQ ID NO: 101 - protein, 10 aa, Homo sapiens
DGAGDVAFVK

SEQ ID NO: 102 - protein, 15 aa, Homo sapiens
LGDTPGVSYSDIAAR

SEQ ID NO: 103 - protein, 19 aa, Homo sapiens
ALLLVGDVAQAADVAIEHR

SEQ ID NO: 104 - protein, 13 aa, Homo sapiens
TVDNFVALATGEK

SEQ ID NO: 105 - protein, 6 aa, Homo sapiens
VYFDLR

SEQ ID NO: 106 - protein, 11 aa, Homo sapiens
FLNLTENDIQK

SEQ ID NO: 107 - protein, 8 aa, Homo sapiens
LPAPSIDR

SEQ ID NO: 108 - protein, 16 aa, Homo sapiens
GKEDFLSVSDIIDYFR

SEQ ID NO: 109 - protein, 15 aa, Homo sapiens
VTFLGLQHWVPELAR

SEQ ID NO: 110 - protein, 8 aa, Homo sapiens
LASHEYLK

SEQ ID NO: 111 - protein, 12 aa, Homo sapiens
AFQVWSDVTPLR

SEQ ID NO: 112 - protein, 19 aa, Homo sapiens
FFGLPQTGDLDQNTIETMR

SEQ ID NO: 113 - protein, 12 aa, Homo sapiens
LTLLAPLNSVFK

SEQ ID NO: 114 - protein, 13 aa, Homo sapiens
GDELADSALEIFK

SEQ ID NO: 115 - protein, 12 aa, Homo sapiens
LPEPATLGFSAR

SEQ ID NO: 116 - protein, 23 aa, Homo sapiens
NPLFDHAALSAPLPAPSSPPALP

SEQ ID NO: 117 - protein, 20 aa, Homo sapiens
SEFVVPDLELPSWLTTGNYR

SEQ ID NO: 118 - protein, 8 aa, Homo sapiens
EGTYSLPK

SEQ ID NO: 119 - protein, 18 aa, Artificial sequence
  Corresponding heavy isotope-labelled peptide internal standard sequence
  MISC_FEATURE (17)..(19): U-13C6,15N4
ALWGKVNVDEVGGEALGR

SEQ ID NO: 120 - protein, 21 aa, Artificial sequence
  Corresponding heavy isotope-labelled peptide internal standard sequence
  MISC_FEATURE (20)..(22): U-13C6,15N2
LLLQKQLSLPETGELDSATLK

SEQ ID NO: 121 - protein, 19 aa, Artificial sequence
  Corresponding heavy isotope-labelled peptide internal standard sequence
  MISC_FEATURE (18)..(20): U-13C6,15N4
NPNLKTLLSVGGWNFGSQR

SEQ ID NO: 122 - protein, 18 aa, Artificial sequence
  Corresponding heavy isotope-labelled peptide internal standard sequence
  MISC_FEATURE (12)..(14): U-13C6,15N4
FSNTDYAVGYMLRLGAPA

SEQ ID NO: 123 - protein, 16 aa, Artificial sequence
  Corresponding heavy isotope-labelled peptide internal standard sequence
  MISC_FEATURE (10)..(12): U-13C6,15N2
ALNSIIDVYHKYSLIK

SEQ ID NO: 124 - protein, 13 aa, Artificial sequence
  Corresponding heavy isotope-labelled peptide internal standard sequence
  MISC_FEATURE (12)..(14): U-13C6,15N4
YSLIKGNFHAVYR

SEQ ID NO: 125 - protein, 20 aa, Artificial sequence
  Corresponding heavy isotope-labelled peptide internal standard sequence
  MISC_FEATURE (14)..(16): U-13C6,15N2
VIEHIMEDLDTNADKQLSFE

SEQ ID NO: 126 - protein, 13 aa, Artificial sequence
  Corresponding heavy isotope-labelled peptide internal standard sequence
  MISC_FEATURE (7)..(9): U-13C6,15N2
LTWASHEKMHEGD

SEQ ID NO: 127 - protein, 15 aa, Artificial sequence
  Corresponding heavy isotope-labelled peptide internal standard sequence
  MISC_FEATURE (14)..(16): U-13C6,15N4
FSGPRYYAFDLIAQR

SEQ ID NO: 128 - protein, 14 aa, Artificial sequence
  Corresponding heavy isotope-labelled peptide internal standard sequence
  MISC_FEATURE (8)..(10): U-13C6,15N2
NTLIIYLDKVSHSE

SEQ ID NO: 129 - protein, 13 aa, Artificial sequence
  Corresponding heavy isotope-labelled peptide internal standard sequence
  MISC_FEATURE (7)..(9): U-13C6,15N4
SQLVYQSRRGPLV

SEQ ID NO: 130 - protein, 21 aa, Artificial sequence
  Corresponding heavy isotope-labelled peptide internal standard sequence
  MISC_FEATURE (20)..(22): U-13C6,15N2
VVHAKGAGAFGYFEVTHDITK

SEQ ID NO: 131 - protein, 21 aa, Artificial sequence
  Corresponding heavy isotope-labelled peptide internal standard sequence
  MISC_FEATURE (15)..(17): U-13C6,15N4
AGLAASLAGPHSIVGRAVVVH

SEQ ID NO: 132 - protein, 14 aa, Artificial sequence
  Corresponding heavy isotope-labelled peptide internal standard sequence
  MISC_FEATURE (8)..(10): U-13C6,15N2
QEILAALEKGCSFL

SEQ ID NO: 133 - protein, 14 aa, Artificial sequence
  Corresponding heavy isotope-labelled peptide internal standard sequence
  MISC_FEATURE (13)..(15): U-13C6,15N2
YYKVEPLDFGGTQK

SEQ ID NO: 134 - protein, 14 aa, Artificial sequence
  Corresponding heavy isotope-labelled peptide internal standard sequence
  MISC_FEATURE (8)..(10): U-13C6,15N2
LSPEDYTLKVSQAG

SEQ ID NO: 135 - protein, 18 aa, Artificial sequence
  Corresponding heavy isotope-labelled peptide internal standard sequence
  MISC_FEATURE (17)..(19): U-13C6,15N4
LTNLKWGLAWVSSQFYNK

SEQ ID NO: 136 - protein, 16 aa, Artificial sequence
  Corresponding heavy isotope-labelled peptide internal standard sequence
  MISC_FEATURE (10)..(12): U-13C6,15N2
ALQATVGNSYKCNAEE

SEQ ID NO: 137 - protein, 18 aa, Artificial sequence
  Corresponding heavy isotope-labelled peptide internal standard sequence
  MISC_FEATURE (12)..(14): U-13C6,15N4
DDILPMDLGTFYREFQNP

SEQ ID NO: 138 - protein, 14 aa, Artificial sequence
  Corresponding heavy isotope-labelled peptide internal standard sequence
  MISC_FEATURE (8)..(10): U-13C6,15N4
QDTLPEAGRRSEAE

SEQ ID NO: 139 - protein, 16 aa, Artificial sequence
  Corresponding heavy isotope-labelled peptide internal standard sequence
  MISC_FEATURE (10)..(12): U-13C6,15N2
QIDLVDNYFVKDTSEA

SEQ ID NO: 140 - protein, 13 aa, Artificial sequence
  Corresponding heavy isotope-labelled peptide internal standard sequence
  MISC_FEATURE (7)..(9): U-13C6,15N4
LAVQFTNRNQYCY

SEQ ID NO: 141 - protein, 12 aa, Artificial sequence
  Corresponding heavy isotope-labelled peptide internal standard sequence
  MISC_FEATURE (11)..(13): U-13C6,15N4
EDYGKGVIFYLR

SEQ ID NO: 142 - protein, 15 aa, Artificial sequence
  Corresponding heavy isotope-labelled peptide internal standard sequence
  MISC_FEATURE (9)..(11): U-13C6,15N4
ENITIVDISRKINQL

SEQ ID NO: 143 - protein, 17 aa, Artificial sequence
  Corresponding heavy isotope-labelled peptide internal standard sequence
  MISC_FEATURE (11)..(13): U-13C6,15N2
IDSLMDEISFLKKVHEE

SEQ ID NO: 144 - protein, 15 aa, Artificial sequence
  Corresponding heavy isotope-labelled peptide internal standard sequence
  MISC_FEATURE (14)..(16): U-13C6,15N4
EQQNKVLEAELLVLR

SEQ ID NO: 145 - protein, 15 aa, Artificial sequence
  Corresponding heavy isotope-labelled peptide internal standard sequence
  MISC_FEATURE (9)..(11): U-13C6,15N2
FTVLTESAAKNTDAV

SEQ ID NO: 146 - protein, 25 aa, Artificial sequence
  Corresponding heavy isotope-labelled peptide internal standard sequence
  MISC_FEATURE (19)..(21): U-13C6,15N4
YPGPQAEGDSEGLSQGLVDREKGLS

SEQ ID NO: 147 - protein, 15 aa, Artificial sequence
  Corresponding heavy isotope-labelled peptide internal standard sequence
  MISC_FEATURE (14)..(16): U-13C6,15N4
SVFSKFIPLIPIPER

SEQ ID NO: 148 - protein, 15 aa, Artificial sequence
  Corresponding heavy isotope-labelled peptide internal standard sequence
  MISC_FEATURE (14)..(16): U-13C6,15N2
NLDLKSDLYIGGVAK

SEQ ID NO: 149 - protein, 20 aa, Artificial sequence
  Corresponding heavy isotope-labelled peptide internal standard sequence
  MISC_FEATURE (14)..(16): U-13C6,15N2
TTVVAAAAFLDAFQKVADMA

SEQ ID NO: 150 - protein, 19 aa, Artificial sequence
  Corresponding heavy isotope-labelled peptide internal standard sequence
  MISC_FEATURE (18)..(20): U-13C6,15N2
LANPRKPVEHWHQLVEVSR

SEQ ID NO: 151 - protein, 19 aa, Artificial sequence
  Corresponding heavy isotope-labelled peptide internal standard sequence
  MISC_FEATURE (13)..(15): U-13C6,15N4
AATVGSAGQPLQERAQAWG

SEQ ID NO: 152 - protein, 14 aa, Artificial sequence
  Corresponding heavy isotope-labelled peptide internal standard sequence
  MISC_FEATURE (8)..(10): U-13C6,15N4
LGPLVEQGRVRAAT

SEQ ID NO: 153 - protein, 14 aa, Artificial sequence
  Corresponding heavy isotope-labelled peptide internal standard sequence
  MISC_FEATURE (8)..(10): U-13C6,15N4
IPTTFENGRCIQAN

SEQ ID NO: 154 - protein, 28 aa, Artificial sequence
  Corresponding heavy isotope-labelled peptide internal standard sequence
  MISC_FEATURE (22)..(24): U-13C6,15N2
VTTVASHTSDSDVPSGVTEVVVKLFDSD

SEQ ID NO: 155 - protein, 25 aa, Artificial sequence
  Corresponding heavy isotope-labelled peptide internal standard sequence
  MISC_FEATURE (19)..(21): U-13C6,15N4
EGHLRSILGTLTVEQIYQDRDQFAK

SEQ ID NO: 156 - protein, 17 aa, Artificial sequence
  Corresponding heavy isotope-labelled peptide internal standard sequence
  MISC_FEATURE (11)..(13): U-13C6,15N4
DVHEGQPLLNVKFSNCN

SEQ ID NO: 157 - protein, 14 aa, Artificial sequence
  Corresponding heavy isotope-labelled peptide internal standard sequence
  MISC_FEATURE (8)..(10): U-13C6,15N2
EVVSPQEFKQGEDA

SEQ ID NO: 158 - protein, 17 aa, Artificial sequence
  Corresponding heavy isotope-labelled peptide internal standard sequence
  MISC_FEATURE (15)..(17): U-13C5,15N
  MISC_FEATURE (16)..(18): U-13C5,15N
VGSNKGAIIGLMVGGVV

SEQ ID NO: 159 - protein, 19 aa, Artificial sequence
  Corresponding heavy isotope-labelled peptide internal standard sequence
  MISC_FEATURE (15)..(17): U-13C5,15N
  MISC_FEATURE (16)..(18): U-13C5,15N
VGSNKGAIIGLMVGGVVIA

SEQ ID NO: 160 - protein, 17 aa, Artificial sequence
  Corresponding heavy isotope-labelled peptide internal standard sequence
  MISC_FEATURE (11)..(13): U-13C6,15N2
LVFFAEDVGSNKGAIIG

SEQ ID NO: 161 - protein, 14 aa, Artificial sequence
  Corresponding heavy isotope-labelled peptide internal standard sequence
  MISC_FEATURE (8)..(10): U-13C6,15N2
DDTPMTLPKGSTEQ

SEQ ID NO: 162 - protein, 15 aa, Artificial sequence
  Corresponding heavy isotope-labelled peptide internal standard sequence
  MISC_FEATURE (9)..(11): U-13C6,15N4
QMYPELQIARVEQAT

SEQ ID NO: 163 - protein, 13 aa, Artificial sequence
  Corresponding heavy isotope-labelled peptide internal standard sequence
  MISC_FEATURE (7)..(9): U-13C6,15N4
AWTVEQLRSEQLP

SEQ ID NO: 164 - protein, 15 aa, Artificial sequence
  Corresponding heavy isotope-labelled peptide internal standard sequence
  MISC_FEATURE (9)..(11): U-13C6,15N2
ESWEMNSEEKLEQST

SEQ ID NO: 165 - protein, 17 aa, Artificial sequence
  Corresponding heavy isotope-labelled peptide internal standard sequence
  MISC_FEATURE (11)..(13): U-13C6,15N4
LSELIQPLPLERDCSAN

SEQ ID NO: 166 - protein, 14 aa, Artificial sequence
  Corresponding heavy isotope-labelled peptide internal standard sequence
  MISC_FEATURE (13)..(15): U-13C6,15N2
KDPQKMYATIYELK

SEQ ID NO: 167 - protein, 17 aa, Artificial sequence
  Corresponding heavy isotope-labelled peptide internal standard sequence
  MISC_FEATURE (11)..(13): U-13C6,15N2
MDSTANEVEAVKVHSFP

SEQ ID NO: 168 - protein, 18 aa, Artificial sequence
  Corresponding heavy isotope-labelled peptide internal standard sequence
  MISC_FEATURE (12)..(14): U-13C6,15N2
VTVNYPPTITESKSNEAT

SEQ ID NO: 169 - protein, 15 aa, Artificial sequence
  Corresponding heavy isotope-labelled peptide internal standard sequence
  MISC_FEATURE (9)..(11): U-13C6,15N2
MYYSAVEPTKDIFTG

SEQ ID NO: 170 - protein, 13 aa, Artificial sequence
  Corresponding heavy isotope-labelled peptide internal standard sequence
  MISC_FEATURE (12)..(14): U-13C6,15N2
DLLFKDSAHGFLK

SEQ ID NO: 171 - protein, 24 aa, Artificial sequence
  Corresponding heavy isotope-labelled peptide internal standard sequence
  MISC_FEATURE (18)..(20): U-13C6,15N4
ALLLVGDVAQAADVAIEHRNEAEL

SEQ ID NO: 172 - protein, 18 aa, Artificial sequence
  Corresponding heavy isotope-labelled peptide internal standard sequence
  MISC_FEATURE (12)..(14): U-13C6,15N2
TVDNFVALATGEKGFGYK

SEQ ID NO: 173 - protein, 21 aa, Artificial sequence
  Corresponding heavy isotope-labelled peptide internal standard sequence
  MISC_FEATURE (15)..(17): U-13C6,15N4
GKEDFLSVSDIIDYFRKMPLL

SEQ ID NO: 174 - protein, 20 aa, Artificial sequence
  Corresponding heavy isotope-labelled peptide internal standard sequence
  MISC_FEATURE (19)..(21): U-13C6,15N4
ASSVRVTFLGLQHWVPELAR

SEQ ID NO: 175 - protein, 17 aa, Artificial sequence
  Corresponding heavy isotope-labelled peptide internal standard sequence
  MISC_FEATURE (16)..(18): U-13C6,15N4
DAFARAFQVWSDVTPLR

SEQ ID NO: 176 - protein, 17 aa, Artificial sequence
  Corresponding heavy isotope-labelled peptide internal standard sequence
  MISC_FEATURE (16)..(18): U-13C6,15N2
SGSERLTLLAPLNSVFK

SEQ ID NO: 177 - protein, 28 aa, Artificial sequence
  Corresponding heavy isotope-labelled peptide internal standard sequence
  MISC_FEATURE (26)..(28): U-13C6,15N
  MISC_FEATURE (27)..(29): U-13C5,15N
EMEVRNPLFDHAALSAPLPAPSSPPALP

SEQ ID NO: 178 - protein, 13 aa, Artificial sequence
  Corresponding heavy isotope-labelled peptide internal standard sequence
  MISC_FEATURE (7)..(9): U-13C6,15N2
EGTYSLPKSEFVV